(19) United States
(12) Patent Application Publication
(10) Pub. No.: US 2020/0081008 A1
(43) Pub. Date: Mar. 12, 2020

(54) METHODS FOR CANCER DETECTION

(71) Applicant: PRIME GENOMICS, INC., Santa Cruz, CA (US)

(72) Inventors: Robert FELDMAN, Santa Cruz, CA (US); Melanie MAHTANI, Santa Cruz, CA (US)

(21) Appl. No.: 16/462,699

(22) PCT Filed: Nov. 22, 2017
(86) PCT No.: PCT/US17/63157

§ 371 Date: May 21, 2019

Related U.S. Application Data
(60) Provisional application No. 62/425,549, filed on Nov. 22, 2016.

(51) Int. Cl. G01N 33/574; C40B 30/04

(52) U.S. Cl. CPC G01N 33/57415; G01N 2800/52; C40B 30/04

(57) ABSTRACT

The present disclosure provides methods for cancer detection. The methods can comprise non-invasive detection of a biomarker from a subject. The methods can be used in combination with additional screening methods for greater accuracy of detection.

### CROSS REFERENCE

This application claims the benefit of U.S. Provisional Application No. 62/425,549, filed Nov. 22, 2016, which is incorporated herein by reference in its entirety.

### BACKGROUND

Cancer is a prevalent disease affecting millions of people across the globe. In 2016, an estimated 1,685,210 new cases of cancer will be diagnosed in the United States alone, and 595,690 people will die from the disease. By 2020, 18.2 million Americans, roughly 1 in 19 people, will be cancer patients or cancer survivors, up from 11.7 million (1 in 26) in 2005.

About 1 in 8 women in the United States will develop invasive breast cancer over the course of her lifetime. In 2012, breast cancer accounted for nearly 25% of all cancer diagnoses. An estimated 252,710 new cases of invasive breast cancer and an estimated 63,410 new cases of non-invasive breast cancer are expected to be diagnosed in women in the United States in 2017. About 2,470 new cases of invasive breast cancer are expected to be diagnosed in men in 2017. Survival rates can be increased if cancer diagnosis occurs at an early stage.

### INCORPORATION BY REFERENCE

All publications, patents, and patent applications herein are incorporated by reference to the same extent as if each individual publication, patent, or patent application was specifically and individually indicated to be incorporated by reference.

### SUMMARY

It shall be understood that different aspects of the invention can be appreciated individually, collectively, or in combination with each other. Various aspects of the invention described herein may be applied to any of the particular applications or methods set forth below.

In an aspect, the present disclosure provides a method for determining a health state of a subject. The method can comprise: a) providing a saliva sample from a subject; b) quantifying a sample level of a biomarker from the saliva sample, wherein the biomarker is from an exosome in the saliva sample; c) comparing the sample level of the biomarker to a reference level of the biomarker, wherein the reference level is obtained from a subject having breast cancer; and d) determining a risk score of the subject for breast cancer based on the comparing. In some embodiments, the method further comprises imaging a breast tissue of the subject. In some embodiments, the imaging is performed using a mammogram. In some embodiments, the method further comprises adjusting the risk score of the subject from step e based on the results from the mammogram. In some embodiments, the method further comprises lysing the exosome to release the biomarker prior to step b). In some embodiments, the method further comprises enriching an exosome fraction of the saliva sample prior to the lysing. In some embodiments, the method further comprises stabilizing the exosome fraction following the enriching. In some embodiments, the biomarker is a cell-free nucleic acid. In some embodiments, the cell-free nucleic acid is RNA. In some embodiments, the RNA is mRNA or miRNA. In some embodiments, the mRNA is a transcript of a gene selected from the group consisting of LCE2B, HIST1H4K, ABCA1, ABCA2, TNFRSF10A, AK092120, DTYMK, ALKBH1, MCART1, Hs.161434, and any combination thereof. In some embodiments, quantifying further comprises reverse transcribing the RNA. In some embodiments, quantifying further comprises performing a polymerase chain reaction (PCR). In some embodiments, PCR comprises qPCR. In some embodiments, quantifying further comprises performing sequencing. In some embodiments, sequencing comprises massively parallel sequencing. In some embodiments, determining the risk score of the subject for breast cancer is performed with an accuracy of at least 90%. In some embodiments, determining the risk score of the subject for breast cancer is performed with a specificity of at least 90%. In some embodiments, determining the risk score of the subject for breast cancer is performed with a sensitivity of at least 80%. In some embodiments, the cell-of-origin of the exosome is a breast cell. In some embodiments, the subject has dense breast tissue. In some embodiments, the subject has an ambiguous result from a screening mammogram. In some embodiments, subject is in an age range of 18 to 40. In some embodiments, the biomarker is a transcript of a gene associated with a hallmark of cancer. In some embodiments, the hallmark of cancer is selected from the group consisting of: evading growth suppressor, avoiding immune destruction, promoting replicative immortality, tumor-promoting inflammation, activating invasion and metastasis, inducing angiogenesis, genome instability and mutation, resisting cell death, deregulating cellular energetics, sustaining proliferative signaling, and any combination thereof. In some embodiments, the gene associated with the hallmark of cancer is selected from the group consisting of: LCE2B, HIST1H4K, ABCA2, TNFRSF10A, AK092120, DTYMK, ALKBH1, MCART1, Hs.161434, and any combination thereof. In some embodiments, the gene associated with the hallmark of cancer is selected from the group consisting of: ABCA1, ABCA2, TNFRSF10A, DTYMK, ALKBH1, and any combination thereof. In some embodiments, the biomarker is a transcript of a gene with an expression profile similar to a gene associated with a hallmark of cancer.

In an aspect, the present disclosure provides a method for reducing a number of false-positive or false-negative results for breast cancer. The method can comprise a) providing a biological sample of a subject, wherein the subject is from a population of subjects having a positive, negative, or ambiguous result from a screening mammogram; b) quantifying a sample level of a biomarker in the biological sample of the subject; c) comparing the sample level of the biomarker to a reference level of the biomarker; and d) identifying the result of the screening mammogram as a false-positive or a false-negative for breast cancer based on the results of the comparing. In some embodiments, the biomarker is a cell-free nucleic acid. In some embodiments, the cell-free nucleic acid is RNA. In some embodiments, the RNA is mRNA or miRNA. In some embodiments, the mRNA is a transcript of a gene selected from the group consisting of LCE2B, HIST1H4K, ABCA2, TNFRSF10A, AK092120, DTYMK, ALKBH1, MCART1, Hs.161434, and any combination thereof. In some embodiments, the biomarker is of exosomal origin. In some embodiments, the method further comprises lysing an exosome fraction of the biological sample to release the biomarker prior to step b). In some embodiments, the method further comprises enriching an exosome fraction of the biological sample prior to the lysing. In some embodiments, the method further comprises stabilizing the exosome fraction following the enriching. In some embodiments, the biological sample is saliva. In some embodiments, the identifying is performed with an accuracy of at least 90%. In some embodiments, the identifying is performed with a specificity of at least 90%. In some embodiments, the identifying is performed with a sensitivity of at least 80%. In some embodiments, the cell-of-origin of the exosome is a breast cell. In some embodiments, the subject has dense breast tissue. In some embodiments, the subject has an ambiguous mammogram result. In some embodiments, the biomarker is a transcript of a gene associated with a hallmark of cancer. In some embodiments, the hallmark of cancer can be selected from the group consisting of: evading growth suppressor, avoiding immune destruction, promoting replicative immortality, tumor-promoting inflammation, activating invasion and metastasis, inducing angiogenesis, genome instability and mutation, resisting cell death, deregulating cellular energetics, sustaining proliferative signaling, and any combination thereof. In some embodiments, the gene associated with the hallmark of cancer is selected from the group consisting of: LCE2B, HIST1H4K, ABCA2, TNFRSF10A, AK092120, DTYMK, ALKBH1, MCART1, Hs.161434, and any combination thereof. In some embodiments, the gene associated with the hallmark of cancer is selected from the group consisting of: ABCA1, ABCA2, TNFRSF10A, DTYMK, ALKBH1, and any combination thereof. In some embodiments, the biomarker is a transcript of a gene with an expression profile similar to a gene associated with a hallmark of cancer.

In an aspect, the disclosure provides a method for determining a health state of a subject. The method can comprise a) providing a biological sample of a subject; b) quantifying a sample level of at least two biomarkers in the biological sample of the subject, wherein the at least two biomarkers are selected from the group consisting of LCE2B, HIST1H4K, ABCA2, TNFRSF10A, AK092120, DTYMK, ALKBH1, MCART1, Hs.161434, and any combination thereof; c) comparing the sample level of the at least two biomarkers to a reference level of the two biomarkers; and d) determining a health state of the subject based on the comparing. In some embodiments, the biological sample is a biological fluid. In some embodiments, the biological fluid is saliva. In some embodiments, one of the at least 2 biomarkers is HIST1H4K. In some embodiments, one of the at least 2 biomarkers is TNFRSF10A. In some embodiments, one of the at least 2 biomarkers is ALKBH1. In some embodiments, one of the at least 2 biomarkers is ABCA2. In some embodiments, one of the at least 2 biomarkers is DTYMK. In some embodiments, the quantifying comprises quantifying the sample level of at least nine biomarkers. In some embodiments, the nine biomarkers are LCE2B, HIST1H4K, ABCA2, TNFRSF10A, AK092120, DTYMK, ALKBH1, MCART1, and Hs.161434. In some embodiments, the quantifying comprises quantifying an mRNA transcript of the at least two biomarkers. In some embodiments, the method further comprises lysing an exosome fraction of the biological sample to release the mRNA. In some embodiments, quantifying the sample level of biomarker is performed with an accuracy of at least 90%. In some embodiments, quantifying the sample level of biomarker is performed with a sensitivity of at least about 80%. In some embodiments, quantifying the sample level of biomarker is performed with a specificity of at least 90%. In some embodiments, the at least 2 biomarkers are associated with a hallmark of cancer. In some embodiments, the hallmark of cancer is selected from the group consisting of: evading growth suppressor, avoiding immune destruction, promoting replicative immortality, tumor-promoting inflammation, activating invasion and metastasis, inducing angiogenesis, genome instability and mutation, resisting cell death, deregulating cellular energetics, sustaining proliferative signaling, and any combination thereof. In some embodiments, the at least two biomarkers comprise an expression profile similar to a gene associated with a hallmark of cancer.

In an aspect, the disclosure provides a method for determining a health state of a subject. The method can comprise a) performing a mammogram on a subject; b) obtaining a saliva sample of the subject; c) quantifying a sample level of a biomarker from the saliva sample, wherein the biomarker is of exosomal origin, wherein the biomarker is a transcript of a gene selected from the group consisting of: LCE2B, HIST1H4K, ABCA2, TNFRSF10A, AK092120, DTYMK, ALKBH1, MCART1, Hs.161434, and any combination thereof; d) comparing the sample level of the biomarker to a reference level of the biomarker, wherein the reference level is obtained from a subject having breast cancer; and e) combining the result of the mammogram and the comparing to determine a health state of the subject associated with breast cancer. In some embodiments, the method has a greater accuracy for determining the health state of the subject associated with breast cancer compared with a method lacking the combining step of step e). In some embodiments, the subject has dense breast tissue. In some embodiments, the mammogram gives an ambiguous result for the subject. In some embodiments, the subject is in an age range of 18 to 40. In some embodiments, the transcript is mRNA or miRNA. In some embodiments, the quantifying comprises sequencing.

In an aspect, the disclosure provides a method comprising: a) providing a saliva sample from a subject; b) quantifying a sample level of a biomarker from the saliva sample, wherein the biomarker is a transcript of a gene associated with a hallmark of cancer; c) comparing the sample level of the biomarker to a reference level of the biomarker, wherein the reference level is obtained from a subject having cancer; and d) determining a risk score of the subject for cancer based on the comparing. In some embodiments, the hallmark of cancer is selected from the group consisting of: evading growth suppressor, avoiding immune destruction, promoting replicative immortality, tumor-promoting inflammation, activating invasion and metastasis, inducing angiogenesis, genome instability and mutation, resisting cell death, deregulating cellular energetics, sustaining proliferative signaling, and any combination thereof. In some embodiments, the gene associated with the hallmark of cancer is selected from the group consisting of: LCE2B, HIST1H4K, ABCA2, TNFRSF10A, AK092120, DTYMK, ALKBH1, MCART1, Hs.161434, and any combination thereof. In some embodiments, the gene associated with the hallmark of cancer is selected from the group consisting of: ABCA1, ABCA2, TNFRSF10A, DTYMK, ALKBH1, and any combination thereof. In some embodiments, the biomarker is obtained from an exosome in the saliva. In some embodiments, the method further comprises lysing the exosome prior to step b to release the biomarker from the exosome fraction. In some embodiments, the cell-of-origin of the exosome is a breast cell. In some embodiments, the transcript is RNA. In some embodiments, the RNA is mRNA or miRNA. In some embodiments, the quantifying further comprises reverse transcribing the RNA. In some embodiments, the quantifying further comprises performing a polymerase chain reaction (PCR). In some embodiments, the PCR is qPCR. In some embodiments, the quantifying further comprises performing sequencing. In some embodiments, the sequencing comprises massively parallel sequencing. In some embodiments, determining the risk score of the subject for cancer is performed with an accuracy of at least 90%. In some embodiments, determining the risk score of the subject for cancer is performed with a specificity of at least 90%. In some embodiments, determining the risk score of the subject cancer is performed with a sensitivity of at least 80%. In some embodiments, the cancer is breast cancer. In some embodiments, the subject has dense breast tissue. In some embodiments, the subject has an ambiguous result from a screening mammogram. In some embodiments, the subject is in an age range of 18 to 40. In some embodiments, the method further comprises imaging a breast tissue of the subject. In some embodiments, the imaging is performed using a mammogram. In some embodiments, the method further comprises adjusting the risk score of the subject from step d based on the results from the mammogram.

### BRIEF DESCRIPTION OF THE FIGURES

The novel features of the disclosure are set forth with particularity in the appended claims. A better understanding of the features and advantages of the disclosure will be obtained by reference to the following detailed description that sets forth illustrative embodiments, in which the principles of the disclosure can be utilized, and the accompanying drawings of which:

FIG. 1 illustrates use of a biological sample of a subject (e.g., body fluid such as saliva) with a biomarker assay of the disclosure to detect biomarkers associated with a health condition (e.g., cancer, breast cancer). Data from the biomarker assay can be used to determine a health condition of the subject.

FIG. 2 illustrates use of a biomarker panel of the disclosure in combination with imaging data (e.g., mammogram) for cancer (e.g., breast cancer) detection in a subject. The use of the assay in combination with imaging data can provide a greater accuracy of detection.

FIG. 3 depicts an illustrative workflow of a method of the disclosure for assessing cancer in a subject using a saliva sample.

FIG. 4 illustrates candidate genes that can be part of a biomarker panel of the disclosure.

FIG. 5 is a block diagram that illustrates an example of a computer architecture system.

FIG. 6 is a diagram showing a computer network with a plurality of computer systems, a plurality of cell phones and personal data assistants, and NAS devices.

FIG. 7 is a block diagram of a multiprocessor computer system using a shared virtual address memory space.

FIG. 8 illustrates a computer program product that is transmitted from a geographic location to a user.

FIG. 9 illustrates results of a study to identify biomarkers for breast cancer. The average connectivity values derived from 10 breast cancer subjects and 10 matched and healthy controls are shown.

FIG. 10 illustrates scores obtained from a 9-gene assay performed using qPCR taken from a validation study of 60 subjects.

FIG. 11 illustrates serially-ordered composite gene expression values.

FIG. 12 illustrates results of a secondary validation study for biomarker gene 5.

FIGS. 13A, 13B, 13C, and 13D show results of a RT-qPCR-based secondary validation study for candidate biomarker genes. FIG. 13A shows the results of a RT-qPCR-based secondary validation study for Gene 2. FIG. 13B shows the results of a RT-qPCR-based secondary validation study for Gene 3. FIG. 13C shows the results of a RT-qPCR-based secondary validation study for Gene 7. FIG. 13D shows the results of a RT-qPCR-based secondary validation study for Gene 9.

FIG. 14 shows parameters and results of the biomarker validation study for Gene 2.

FIG. 15 shows parameters and results of the biomarker validation study for Gene 3.

FIG. 16 shows parameters and results of the biomarker validation study for Gene 7.

FIG. 17 shows parameters and results of the biomarker validation study for Gene 9.

FIGS. 18A, 18B, 18C, 18D, and 18E illustrate results of a RT-qPCR-based secondary validation study for candidate biomarker genes. FIG. 18A shows the results of a RT-qPCR-based secondary validation study for Gene 1. FIG. 18B shows the results of a RT-qPCR-based secondary validation study for Gene 4. FIG. 18C shows the results of a RT-qPCR-based secondary validation study for Gene 5. FIG. 18D shows the results of a RT-qPCR-based secondary validation study for Gene 6. FIG. 18E shows the results of a RT-qPCR-based secondary validation study for Gene 8.

FIG. 19 shows parameters and results of the biomarker validation study for Gene 1.

FIG. 20 shows parameters and results of the biomarker validation study for Gene 4.

FIG. 21 shows parameters and results of the biomarker validation study for Gene 5.

FIG. 22 shows parameters and results of the biomarker validation study for Gene 6.

FIG. 23 shows parameters and results of the biomarker validation study for Gene 8.

FIG. 24 shows the results of a RT-qPCR-based secondary validation study for the housekeeping gene G-H1.

FIG. 25 shows the results of a RT-qPCR-based secondary validation study for the housekeeping gene G-H2.

FIG. 26 illustrates an example of an optimized work flow for the saliva biomarker test.

FIG. 27 depicts illustrative genes and signaling systems associated with hallmarks of cancer.

FIG. 28 depicts illustrative biomarkers identified using the methods of the disclosure that are associated with one or more hallmarks of cancer.

FIG. 29 illustrates results of a study to evaluate gene expression profiles in saliva for breast cancer genes.

### DETAILED DESCRIPTION OF THE DISCLOSURE

The following description and examples illustrate embodiments of the disclosure in detail. It is to be understood that this disclosure is not limited to the particular embodiments described herein and as such can vary. Those of skill in the art will recognize that there are numerous variations and modifications of the disclosure, which are encompassed within its scope.

Imaging tests such as mammograms can be used to screen and detect breast diseases like cancer (e.g., invasive breast cancer and ductal carcinoma in situ). However, screening mammograms can fail to detect about 1 in 5 breast cancers. False-positive and false-negative rates for mammograms can range from, for example, about 7-15%. False-positive and false-negative rates can be more frequent in younger women (e.g., women under 50) and women with dense breasts. Further, it can be difficult to differentiate between invasive breast cancer and non-life-threatening forms of breast cancer based on mammograms. Consequently, mammograms can lead to the over-diagnosis of patients, which can lead to overtreatment of cancers that are not invasive. Thus, there exists a considerable need for more accurate methods of breast cancer detection.

Disclosed herein are methods for detecting cancer in a subject. An exemplary method can comprise the steps of (a) obtaining a biological sample of a subject, (b) quantifying a sample level of a biomarker in the biological sample, (c) comparing the sample level of the biomarker to a reference level of the biomarker, (d) determining a risk score of the subject for a cancer based on the comparison between the sample level and the reference level, or any combination thereof. The biological sample can be, for example, saliva. The cancer can be, for example, breast cancer. The biomarker can be, for example, of exosomal origin. The method can additionally comprise a step of lysing, isolating, or enriching a specific fraction of the biological sample, for example, exosomes in the biological sample.

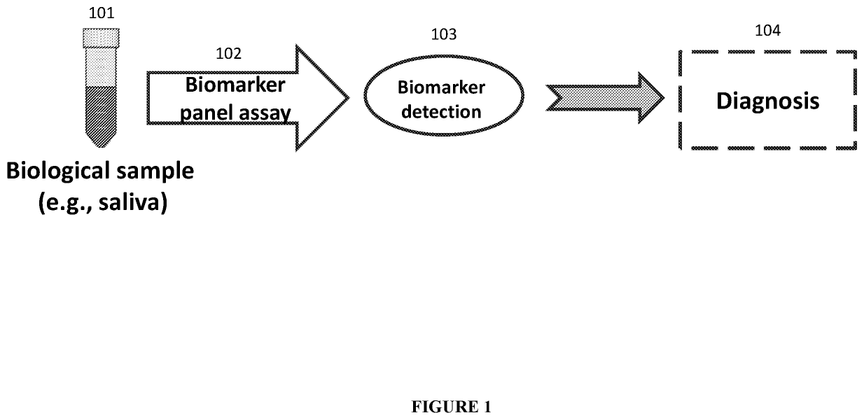

An exemplary method of the disclosure is depicted in FIG. 1. FIG. 1 illustrates use of a saliva sample from a subject to detect one or more biomarkers associated with, for example, cancer. A saliva sample (101) is collected from a subject. The saliva sample is then processed and subjected to a biomarker panel assay of the disclosure (102) to detect biomarkers (103). Results of the biomarker assay are used to determine whether the subject has cancer. The subject is given a diagnosis (104).

A method of the disclosure can be used in combination with an additional screening or detection method. For example, a combination of a biomarker assay of the disclosure and an additional screening test can provide a higher accuracy, sensitivity, and/or specificity of detection of cancer, compared with that obtained using the screening test alone. An exemplary method can comprise the steps of a) performing a screening test on a subject to evaluate a risk of developing a health condition by the subject, b) obtaining a biological sample of the subject, c) quantifying a sample level of a biomarker in the biological sample of the subject, d) comparing the sample level of the biomarker to a reference level of the biomarker, e) combining the result of the screening test and the biomarker comparison, f) determining a health state of the subject based on the combined information from the screening test and the biomarker results, or any combination thereof. The additional screening test can include, for example, an imaging test (e.g., using x-rays, sound waves, radioactive particles, or magnetic fields) from a tissue or organ of the subject. The tissue can be, for example, breast tissue. The additional screening test can be, for example, a mammogram. The biological sample can be, for example, saliva. The cancer can be, for example, breast cancer. The biomarker can be, for example, of exosomal origin. The biomarker can be, for example, mRNA.

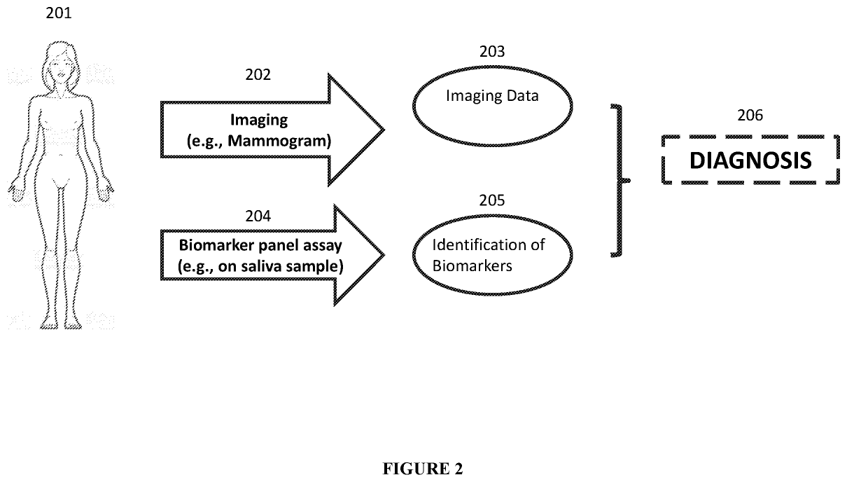

An exemplary method of the disclosure is depicted in FIG. 2. FIG. 2 illustrates the use of a saliva-based biomarker assay of the disclosure in conjunction with an additional screening test (e.g., mammogram) for detecting breast cancer. A subject (201) undergoes an imaging test such as a mammogram (202). The subject also provides a sample such as saliva (204) for a biomarker panel assay. Imaging data (203) are obtained and processed. The saliva sample is subjected to a biomarker panel assay to detect biomarkers (205). A combination of the imaging data (203) and biomarker assay results (205) are used to diagnose breast cancer in the subject (206).

Disclosed herein are methods for reducing the number of false-positive or false-negative results for a health condition. An exemplary method can comprise the steps of a) obtaining a biological sample of a subject with a positive, negative, or ambiguous result from a screening test that evaluates the subject's risk of developing a health condition, b) quantifying a sample level of a biomarker in the biological sample of the subject, c) comparing the sample level of the biomarker to a reference level of the biomarker for the health condition, d) identifying the result of the screening test as a false-positive or a false-negative for the health condition based on the results from the biomarker comparison. The screening test can include, for example, an imaging test (e.g., using x-rays, sound waves, radioactive particles, or magnetic fields) from a tissue or organ of the subject. The tissue can be, for example, breast tissue. The screening test can be, for example, a mammogram. The biological sample can be, for example, saliva. The health condition can be, for example, breast cancer. The biomarker can be, for example, of exosomal origin. The biomarker can be, for example, mRNA.

Methods of the disclosure can provide, for example, a low cost, accurate, non-invasive, and easy to implement test for early detection of cancer. Methods of the disclosure can aid early detection of cancer. Methods of the disclosure can be useful for subjects with dense breast tissue. Methods of the disclosure can reduce the rate of false positives and false negatives, and improve the accuracy of cancer diagnosis. In some embodiments, the disclosure provides a saliva-based test that comprises measuring mRNA of exosomal origin from a saliva sample of the subject to determine the subject's risk of breast cancer.

In some embodiments, the disclosure provides a device for performing the methods of the disclosure. The device can be used to analyze a sample, for example, to generate a biomarker signature of the subject. In some embodiments, the device can be used at a clinic, a hospital, or a breast imaging center.

Aspects of the disclosure can relate to methods that can improve the monitoring, diagnosing, and/or treatment of a subject suffering from a health condition or a disease. The health condition can be, for example, cancer, neurodegenerative diseases, inflammatory disorders, or a drug response disorder.

Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor. In some embodiments, the health condition is cancer. In some embodiments, the health condition is breast cancer.

A method of the disclosure can comprise detecting the presence of a biomarker. A biomarker can be a measurable indicator of a health condition (e.g., cancer). A biomarker can be secreted by a tumor or as a result of a physiological response, for example, from the presence of cancer. A biomarker can be, for example, genetic, epigenetic, proteomic, glycomic, or imaging biomarker. A biomarker can be used for diagnosis, prognosis, or epidemiology. A biomarker can be assayed in an invasively collected sample such as a tissue biopsy. A biomarker can be assayed in a non-invasively collected sample such as bodily fluids, for example, saliva.

Various biomarkers are suitable for use with a method of the disclosure. A biomarker can be, for example, a nucleic acid such as DNA or RNA, a peptide, a protein, a lipid, an antigen, an antibody, a carbohydrate, a proteoglycan, or any combination thereof. A biomarker can be a cell-free nucleic acid, such as cell-free DNA or cell-free RNA. A biomarker can be RNA selected from the group consisting of: mRNA, small RNA, miRNA, snoRNA, snRNA, rRNAs, tRNAs, siRNA, hnRNA, shRNA, and a combination thereof. In some embodiments, a biomarker can be RNA. In some embodiments, a biomarker can be mRNA. In some embodiments, a biomarker can be miRNA.

A biomarker can be a product (e.g., expression product) of a gene. A biomarker can measure the activity of a gene. The expression of a biomarker gene can be measured at a transcriptomic level (e.g., RNA, mRNA, miRNA), proteomic level (e.g., protein, polypeptide), or a combination thereof.

A biomarker gene can be differentially expressed (e.g., overexpressed or under-expressed), for example, in comparison to a reference level or control for a health condition. For example, a biomarker can have a change in expression level of at least 2-fold, 3-fold, 4-fold, 5-fold, 6-fold, 7-fold, 10-fold, 15-fold, 20-fold, 50-fold change, or 100 fold compared with a reference level for a health condition. In some embodiments, the difference in gene expression level is at least 10%, 15%, 20%, 25%, 30%, 35%, 40%, 45% or 50% or more. A reference level can be obtained from one or more subjects. A method of the disclosure can comprise determining the differential expression of a biomarker gene compared with a control.

A method of the disclosure can comprise detection of more than one biomarker. A method can assess, for example, at least 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 400, 500, 600, 700, 800, 900, or 1000 biomarkers. A method can assess, for example, 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 400, 500, 600, 700, 800, 900, or 1000 biomarkers. A method can comprise detecting at least 2 biomarkers. A method can comprise detecting at least 3 biomarkers. A method can comprise detecting at least 4 biomarkers. A method can comprise detecting at least 5 biomarkers. A method can comprise detecting at least 6 biomarkers. A method can comprise detecting at least 7 biomarkers. A method can comprise detecting at least 8 biomarkers. A method can comprise detecting at least 9 biomarkers.

Detection or analysis of a biomarker can comprise determination of: an expression level, presence, absence, mutation, copy number variation, truncation, duplication, insertion, modification, sequence variation, molecular association, or a combination thereof, of the biomarker.

In some embodiments, gene co-expression networks can be analyzed to discover biomarkers. See also e.g., U.S. Patent Publication 20120010823, which is incorporated herein by reference in its entirety for all purposes. Analysis of gene co-expression networks can be based on the transcriptional response of cells to changing conditions. Because the coordinated co-expression of genes can encode interacting proteins, studying co-expression patterns can provide insights into the underlying cellular processes.

A threshold can be set on a Pearson correlation coefficient to arrive at gene co-expression networks, which can be referred to as ‘relevance’ networks. In these networks, a node can correspond to the gene expression profile of a given gene. Nodes can be connected, for example, if they have a significant pairwise expression profile. In some embodiments, the absolute value of a Pearson correlation can be used as a standard in a gene expression cluster analysis. In some embodiments, the Pearson correlation coefficient can be used as a co-expression measure.

Methods of the disclosure can comprise analysis of gene expression modules. A clustering procedure can be used to identify modules of connected nodes with a high correlation, for example, greater than 0.95, between their gene expression values. Average connectivity between these modules can then be analyzed. The average connectivity can be the average of the kᵢ across all the modules. Connectivity for a module i can be defined as the kᵢ modules linked with, for example, greater than about 0.95 correlation to module i:

$$ki = \sum {a}_{ij}$$

$$i \neq j$$

where aᵢⱼ can be a module with a correlation greater than 0.95 to the ith module

In some embodiments, gene expression values can be weighted. In some embodiments, new and/or additional genes can be added to the biomarker panel. In some embodiments, weighting of gene expression values and/or additional genes can improve scoring of subjects, which can lead to greater accuracy of biomarker detection. Improved scoring can lead to increased sensitivity, for example, greater than 90%. In some cases, a weighting regime may not be used.

Identified biomarker genes can exhibit differences in connectivity or co-expression between the subjects with a health condition such as breast cancer, and healthy subjects. This can be occur, for example, when moving from examining gene expression output from a gene chip during discovery phase studies to examining gene expression output using qPCR, which can have a greater dynamic range and sensitivity. A measure of average connectivity within the gene sub-network can be used to score qPCR results and indicate differences between breast cancer subjects and healthy subjects. Comparing the average connectivity within a gene sub-network can provide data that allows weighting of gene expression values, or add new genes, to improve the scores for greater accuracy of the test.

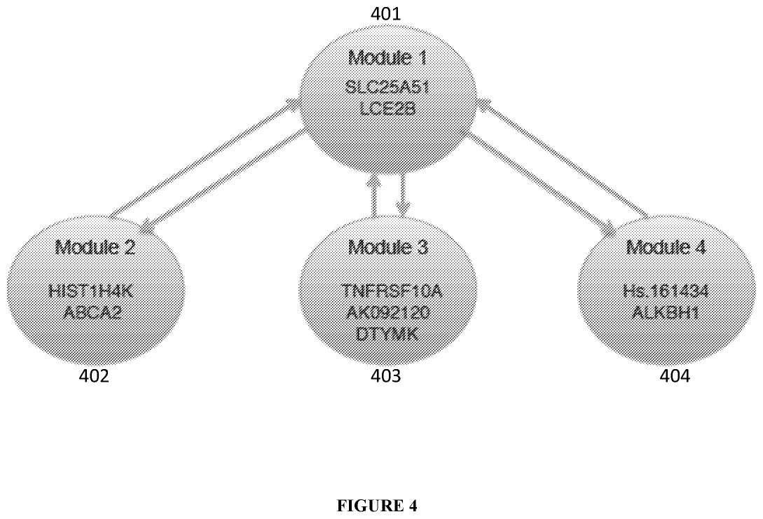

FIG. 4 and TABLE 1 show illustrative biomarkers identified using methods of the disclosure. One or more of these biomarkers can be a part of a biomarker panel. A “biomarker panel”, “biomarker gene panel”, or “biomarker assay panel” can refer to a set of biomarkers that can be analyzed in a biological sample to determine a health state of the subject or risk for a health condition such as breast cancer. In some cases, a subset or variant of a panel can be used.

TABLE 1

| | | GenBank | |
|---|---|---|---|
| | | Accession | |
| Probe ID | Gene Symbol | Number | Notes |
| 238574_at | MCART1 | BF724944 | |
| 207710_at | LCE2B | NM_014357 | |
| 243218_at | Hs.161434 | AI424847 | |
| 205621_at | ALKBH1 | NM_006020 | |
| 208580_x_at | HIST1H4A /// | NM_021968 | |
| | HIST1H4B /// | | |
| | HIST1H4C /// | | |
| | HIST1H4D /// | | |
| | HIST1H4E /// | | |
| | HIST1H4F /// | | |
| | HIST1H4H /// | | |
| | HIST1H4I /// | | |
| | HIST1H4J /// | | |
| | HIST1H4K /// | | |
| | HIST1H4L /// | | |
| | HIST2H4A /// | | |
| | HIST2H4B /// HIST4H4 | | |
| 212772_s_at | ABCA2 | AL162060 | |
| 241371_at | Hs.57851 | AW451259 | TNFRSF10A |
| 1566840_at | LOC283674 | AK092120 | |
| 1565694_at | DTYMK /// LOC727761 | AK022132 | |
| Normalizing | | | |
| genes | | | |
| 212686_at | PPM1H | AB032983 | |
| | TFRC | | |
| | ACTB | | |

A biomarker panel can comprise at least 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 400, 500, 600, 700, 800, 900, or 1000 biomarkers. A biomarker panel can comprise 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 400, 500, 600, 700, 800, 900, or 1000 biomarkers. A biomarker panel can comprise at least 2 biomarkers. A biomarker panel can comprise at least 3 biomarkers. A biomarker panel can comprise at least 4 biomarkers. A biomarker panel can comprise at least 5 biomarkers. A biomarker panel can comprise at least 6 biomarkers. A biomarker panel can comprise at least 7 biomarkers. A biomarker panel can comprise at least 8 biomarkers. A biomarker panel can comprise at least 9 biomarkers. A biomarker panel can comprise at least 10 biomarkers.

A biomarker can be, for example, a cancer-related or cancer-associated gene, a gene in a breast cancer pathway, an oncogene, a gene associated with or implicated in a hallmark of cancer, or a combination thereof. A biomarker can be selected from the group consisting of: MCART1, LCE2B, HIST1H4K, ABCA1, ABCA2, ABCA12, TNFRSF10A, AK092120, DTYMK, Hs.161434, ALKBH1, and homologs, variants, derivatives, product, and combinations thereof. A biomarker can be a homolog, variant, derivative, or product of a gene disclosed herein. It will be understood that the disclosure covers other names and aliases of genes disclosed herein.

In some embodiments, the biomarker can be MCART1. In some embodiments, the biomarker can be LCE2B. In some embodiments, the biomarker can be HIST1H4K. In some embodiments, the biomarker can be ABCA1. In some embodiments, the biomarker can be ABCA2. In some embodiments, the biomarker can be TNFRSF10A. In some embodiments, the biomarker can be AK092120. In some embodiments, the biomarker can be DTYMK. In some embodiments, the biomarker can be Hs.161434. In some embodiments, the biomarker can be ALKBH1.

In some embodiments, a biomarker panel comprises at least 2 biomarkers, for example, HIST1H4K and TNFRSF10A. In some embodiments, a biomarker panel comprises at least 2 biomarkers, for example, HIST1H4K and TNFRSF10A. In some embodiments, a biomarker panel can comprise at least 2, 3, 4, 5, 6, 7, 8, or 9 biomarkers selected from the group consisting of: MCART1, LCE2B, HIST1H4K, ABCA1, ABCA2, ABCA12, TNFRSF10A, AK092120, DTYMK, ALKBH1, Hs.161434, and variants thereof.

A biomarker can be a gene or a gene product of the solute carriers (SLC) gene family, for example, MCART1. MCART1 can also be known as SLC25A51, CG7943, or MGC14836. MCART1 can be found on chromosome 9 with a chromosome location (bp) of 37879400-37904353. MCART1 can be differentially expressed in cancer (e.g., breast cancer). Mutations such as amplification in, for example, the region of chromosome 9 (e.g., 9p13.3-p13.2) in breast cancer can be associated with overexpression of MCART1. SLC25 is a large family of nuclear-encoded transporters embedded in the inner mitochondrial membrane and other organelle membranes. Members of the SLC25 superfamily can be involved in numerous metabolic pathways and cell functions. SLC25 family members can be recognized by their sequence features such as a tripartite structure, six transmembrane α-helices, and a 3-fold repeated signature motifs. SLC25 members vary greatly in the nature and size of their transported substrates, modes of transport (i.e., uniport, symport, or antiport) and driving forces. Mutations in the SLC25 genes can be associated with various disorders related to, for example, carnitine/acylcarnitine carrier deficiency, hyperonithinemia-hyperammonemia-homocitrullinuria syndrome, aspartate/glutamate isoform 1 and 2 deficiencies, congenital Amish microcephaly, neuropathy with bilateral striatal necrosis, congenital sideroblastic anemia, neonatal epileptic encephalopathy, and citrate carrier deficiency.

A biomarker can be late cornified envelope 2B (LCE2B) or a product thereof. LCE2B can also be known as small proline-rich-like epidermal differentiation complex protein 1B (SPRL1B), skin-specific protein Xp5 (XP5), and late envelope protein 10 (LEP10). LCE2B can be located on chromosomal band 1q21. LCE2B can be involved in epidermal differentiation. Pathways related to LCE2B can be, for example, keratinization, cytokine inflammation, and host response to bacteria. A paralog of LCE2B gene that can also serve as a biomarker is LCE2C.

A biomarker can be a gene or gene product in the Histone cluster 1 H4 family, for example, Histone cluster 1 H4 member K (HIST1H4K), which can also be known as H4 histone family-member D, histone cluster 1-H4k, H4/D, H4FD, histone H4, or DJ160A22.1. Histones can be basic nuclear proteins that are responsible for the nucleosome structure of the chromosomal fiber, and for transcriptional activation of genes in cancer. Two molecules of each of the four core histones (H2A, H2B, H3, and H4) can form an octamer, around which approximately 146 bp of DNA can be wrapped in repeating units, called nucleosomes. The linker histone, H1, can interact with linker DNA between nucleosomes and function in the compaction of chromatin into higher order structures. HIST1H4K can be intronless and can encode a replication-dependent histone that is a member of the histone H4 family, histone H4. Transcripts from HIST1H4K can lack polyA tails and may contain a palindromic termination element. HIST1H4K can be found in the small histone gene cluster on chromosome 6p22-p21.3.

A biomarker can be a gene or gene product of the ATP binding cassette (ABC) family, for example, ATP binding cassette subfamily A member 2 (ABCA2), which can also be known as ATP-binding cassette transporter 2, ATP-binding cassette 2, ABC2, EC 3.6.3.41, KIAA1062, and EC 3.6.3. ABC proteins can transport various molecules across extra- and intracellular membranes. Proteins encoded by the ABC subfamily can be highly expressed in, for example, brain tissue and may play a role in macrophage lipid metabolism and neural development. ABC genes can be divided into seven subfamilies: ABC1, MDR/TAP, MRP, ALD, OABP, GCN20, and White. A biomarker can be, for example, ABCA1, ABCA2, ABCA3, ABCA4, ABCA7, ABCA12, or ABCA13. ABCA2 can be a member of the ABC1 subfamily. ABCA2 can encode, for example, two transcript variants. Overexpression of ABC transporters can offer an adaptive advantage used by tumor cells to evade the accumulation of cytotoxic agents. For example, ABCA2, which can be highly expressed in the cells of the nervous and haematopoetic systems, can be associated with lipid transport and drug resistance in cancer cells including tumor stem cells.

A biomarker can be a gene or gene product of the Tumor necrosis factor receptor superfamily, for example, Tumor necrosis factor receptor superfamily member 10A (TNFRSF10A), which can also be known as TNF-related apoptosis-inducing ligand receptor 1, death receptor 4, TRAIL receptor 1 (TRAILR-1), APO2, DR4, and CD261 antigen. TNF receptors can be activated by TNF-related apoptosis inducing ligand (TNFSF10/TRAIL), which can transduce cell death signaling and induce cell apoptosis. Fas-associated protein with death domain FADD, a death domain containing adaptor protein, can be required for apoptosis mediated by TNF receptor protein. The adapter molecule FADD can recruit caspase-8 to the activated receptor. The resulting death-inducing signaling complex (DISC) can perform caspase-8 proteolytic activation which can initiate the subsequent cascade of caspases (e.g., aspartate-specific cysteine proteases) mediating apoptosis. TNFRSF10A can promote activation of NF-kappa-B. Diseases associated with TNFRSF10A can include posterior scleritis and pharyngoconjunctival fever. TNFRSF10A can be associated with TRAF pathway, apoptosis, and autophagy. A paralog of TNFRSF10A can be TNFRSF10B, which can also be used as a biomarker herein.

A biomarker can be AK092120. AK092120 can be related to LOC283674. AK092120 can be, for example, an miRNA or a transcription binding site. AK092120 can be associated with, correlated with, surrogate for, or behaving similar (e.g., similarly expressed) to a gene associated with a hallmark of cancer. AK092120 can be a hallmark of cancer gene.

A biomarker can be deoxythymidylate kinase (DTYMK), which can also be known as thymidylate kinase, CDC8, TMPK, TYMK, EC 2.7.4.9, and PP3731. Among DTYMK's related pathways can be the superpathways of pyrimidine deoxyribonucleotide de novo biosynthesis and purine metabolism (KEGG pathway). DTYMK can be involved in, for example, kinase activity and thymidylate kinase activity. The protein encoded by DTYMK can catalyze the conversion of deoxythymidine monophosphate (dTMP) to deoxythymidine diphosphate (dTDP). A deficiency in DTYMK can be associated with decreased growth and lethality in cancer cells.

A biomarker can be Hs. 161434. Hs. 161434 can be, for example, an miRNA or a transcription binding site. Hs. 161434 can be associated with, correlated with, surrogate for, or behaving similar (e.g., similarly expressed) to a hallmark of cancer gene. Hs. 161434 can be a hallmark of cancer gene.

A biomarker can be a gene or gene product of the AlkB family, for example, AlkB homolog 1 (ALKBH1), which belong to the 2-oxoglutarate and Fe2+ dependent hydroxylase family. ALKBH1 is a histone dioxygenase that can remove methyl groups from histone H2A. ALKBH1 can be a gene associated with a hallmark of cancer. It can act on nucleic acids, such as DNA, RNA, tRNA. It can act as a regulator of translation initiation and elongation, for example, in response to glucose deprivation. ALKBH1 can be a demethylase for DNA N6-methyladenine (N6-mA), an epigenetic modification, and can interact with the core transcriptional pluripotency network of embryonic stem cells. Expression of ALKBH1 in human mesenchymal stem cells (MSCs) can be upregulated in stem cell induction. Depletion of ALKBH1 can result in the accumulation of N6-mA on the promoter region of activating transcription factor 4 (ATF4), which can silence ATF4 transcription. ALKBH1 can be involved in reversible methylation of tRNA, which can serve as a mechanism of post-transcriptional gene expression regulation.

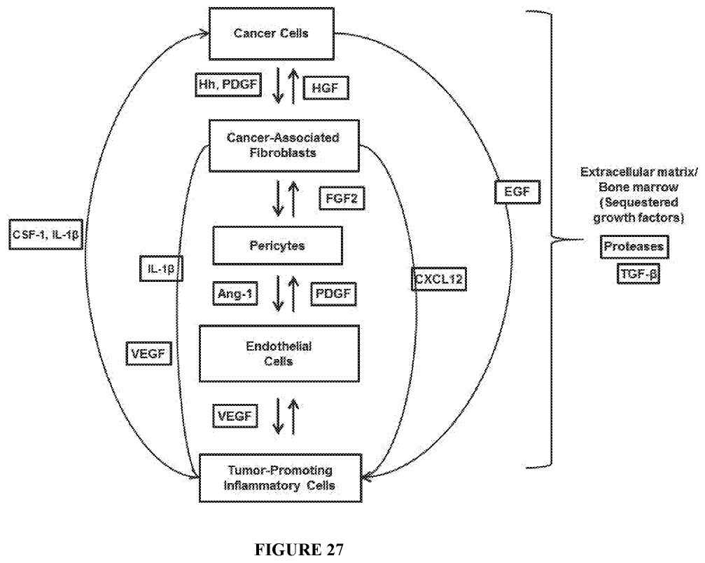

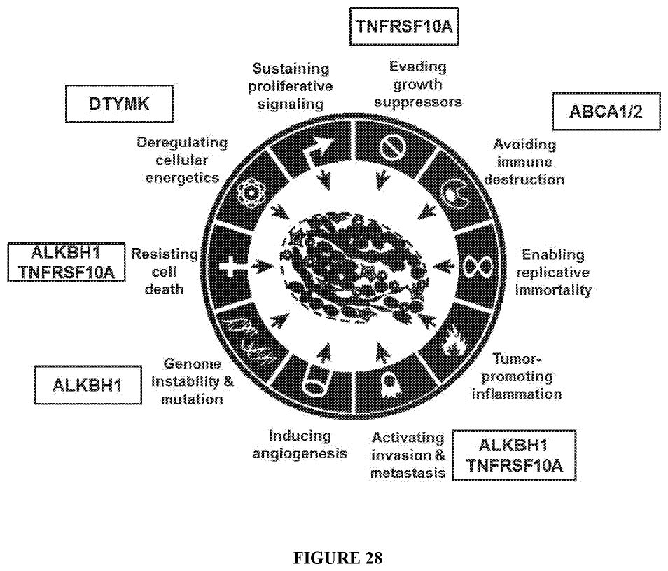

A biomarker can be a gene associated with a hallmark of cancer (see e.g., Hanahan D and Weinberg R A (January 2000) Cell. 100 (1): 57-70 and Hanahan, D and Weinberg, R. A. (2011) Cell. 144 (5): 646-674, which are incorporated herein by reference in their entirety for all purposes). A gene disclosed herein, such as shown in FIG. 4 or TABLE 1, can be a hallmark of cancer gene. A hallmark of cancer gene or a hallmark gene can be a gene associated with a hallmark of cancer. Cancers can have hallmarks, which can govern transformation of normal cells to malignant or tumor cells. The traits or hallmarks can be, for example, self-sufficiency in growth signals, insensitivity to anti-growth signals, evading apoptosis, limitless replicative potential, sustained angiogenesis, tissue invasion, metastasis, abnormal metabolic pathways, evading the immune system, genome instability, and inflammation. Cancer microenvironments can require signaling systems that utilize hallmark genes for tumor growth. FIG. 27 illustrates exemplary hallmark of cancer genes and signaling systems. FIG. 28 illustrates biomarkers identified using the methods of the disclosure, such as DTYMK, TNFRSF10A, ABCA1/2 and ALKBH1, that are associated with one or more hallmarks of cancer. A method can comprise determining differential expression of a gene associated with a hallmark of cancer. A method can comprise determining differential expression of a gene that is a surrogate of (e.g., correlated with or having similar expression profile to) a gene associated with a hallmark of cancer.

A biomarker can be a gene associated with, correlated with, surrogate or substitute for, or that behaves similarly (e.g., similarly expressed, correlated) to a hallmark of cancer gene. For example, a gene disclosed herein, such as shown in FIG. 4 or TABLE 1, can be correlated to or have an expression profile similar to a hallmark of cancer gene, and can therefore be used as a substitute or proxy for a hallmark gene.

A biomarker can be a gene associated with a breast cancer pathway. Non-limiting examples of such genes include ABL1, AHR, AKT1, ANXA1, AR, ARAF, ATF1, ATM, ATR, BACH1, BAD, BAK1, BARD1, BAX, CCND1, BCL2, BID, BLM, BMPR1A, BMPR2, BRCA1, BRAF, BRCA2, CASP3, CASP8, CASP9, CDC25A, CDC25B, CDC42, CDH1, CDK2, CDK4, CDK7, CHEK1, CHUK, PLK3, CREB1, CSNK1D, CTNNB1, CYP19A1, DAG1, GADD45A, E2F1, EGFR, EP300, ESR1, FAU, FER, FOXO1, MTOR, GDI1, GRN, GSK3A, MSH6, HDAC1, HMGCR, IMPA1, IRS1, JAK1, JUN, KRAS, SMAD1, SMAD2, SMAD4, SMAD6, SMAD7, MAX, MDM2, MMP1, MRE11, MSH2, MYC, MYT1, NAB1, NF1, NFKB1, ODC1, PAK1, PHB, PIGR, PIK3R2, PLK1, PML PKIA, MAPK1, PTEN, RAC1, RAD51, RALA, RAP1A, RB1, RHEB, RHO, RRAS, SMARCA4, SP1, STAT1, AURKA, STK11, TFPI, TGFBR1, TGFBR2, TP53, TPR, TSC1, TSC2, VEGFA, WEE1, XRCC3, FOSL1, NCOA3, RAD54L, PIAS1, TRADD, FADD, ALKBH1, MAP3K13, USP15, RAD50, TAB1, RPP38, USP16, NOXA1, EDAR, CHEK2, MYCBP2, SIRT1, ZMYND8, RASGRP3, ERAL1, USP21, FILIP1, HIPK2, LGALS13, DHTKD1, PPP4R3A, MAP3K7CL, ZMIZ1, PPP4R3B, CCNB1IP1, APOBEC3G, CERK, ZNF655, DCAKD, NUP85, ITPKC, USP38, UBE2F, JAKMIP1, RASGEF1A, RALGAPA1, MIR1281, GRIK1-AS2, or any combination thereof.

A set of biomarkers can be customized based on, for example, specific breast cancer subtypes, disease severity, genes that can predict disease treatment types or modalities, or a combination thereof. A biomarker panel can include, for example, at least 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 400, 500, 600, 700, 800, 900, or 1000 biomarkers. In some embodiments, a biomarker panel can comprise at least 9 biomarkers.

A method of the disclosure can comprise obtaining or providing a biological sample from a subject. A biological sample can be any substance containing or presumed to contain a biomarker. A biological sample can be any substance containing or presumed to contain a nucleic acid or protein.

The biological sample can be a liquid sample. The biological sample can be a body fluid. The biological sample can be a sample that comprises exosomes. The biological fluid can be an essentially cell-free liquid sample, for example, saliva, plasma, serum, sweat, urine, and tears. In other embodiments, the biological sample can be a solid biological sample, e.g., feces or tissue biopsy, e.g., a tumor biopsy. A sample can also comprise in vitro cell culture constituents including but not limited to conditioned medium resulting from the growth of cells in cell culture medium, recombinant cells and cell components.

A biological sample can be selected from the group consisting of: blood, serum, plasma, urine, sweat, tears, saliva, sputum, components thereof and any combination thereof. In some embodiments, a biological sample can be saliva. In some embodiments, a biological sample can be blood.

Non-limiting examples of a biological sample include saliva, whole blood, peripheral blood, plasma, serum, ascites, cerebrospinal fluid, sweat, urine, tears, buccal sample, cavity rinse, sputum, organ rinse, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample can also include the blastocyl cavity, umbilical cord blood, or maternal circulation which may be of fetal or maternal origin. The biological sample may also be a tissue sample or biopsy, from which exosomes may be obtained. For example, if the sample is a solid sample, cells from the sample can be cultured and exosome product induced or retrieved.

Collection of a biological sample can be performed in any suitable setting, for example, hospitals, home, clinics, pharmacies, breast imaging clinics, and diagnostic labs. A biological sample can be transported by mail or courier to a central clinic for analysis. A biological sample can be stored under suitable conditions prior to analysis.

A method of the disclosure can be used to detect a biomarker from an exosome. For example, a biomarker from an exosomal fraction of a biological sample or a biomarker of exosomal origin.

Exosomes can be small membrane bound vesicles that can be released into the extracellular environment from a variety of different cells such as but not limited to, cells that originate from, or are derived from, the ectoderm, endoderm, or mesoderm including any such cells that have undergone genetic, environmental, and/or any other variations or alterations (e.g. bacterial/virally infected cells, tumor cells or cells with genetic mutations). An exosome can be created intracellularly when a segment of the cell membrane spontaneously invaginates and is ultimately exocytosed.

Exosomes can have a diameter of about 30-1000 nm, about 30-800 nm, about 30-200 nm, about 30-100 nm, about 20 nm to about 100 nm, about 30 nm to about 150 nm, about 30 nm to about 120 nm, about 50 nm to about 150 nm, or about 50 nm to about 120 nm.

Exosomes can also be referred to as microvesicles, nanovesicles, vesicles, dexosomes, bleb, blebby, prostasomes, microparticles, intralumenal vesicles, endosomal-like vesicles or exocytosed vehicles. Exosomes can also include any shed membrane bound particle that is derived from either the plasma membrane or an internal membrane. Exosomes can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen, including but not limited to tumor-derived microRNAs or intracellular proteins.

An exosome can be a source of a biomarker. Exosomes can be present in, for example, biological fluids such as saliva, blood, urine, cerebrospinal fluid, and breast milk. Exosomes can comprise proteins and nucleic acids. All cell types in culture can secrete exosomes. Exosomes can be involved in intercellular signaling. Exosomes can contain molecular constituents of their cell of origin, i.e. a cell from which the exosome originated. There can be a correlation between exosomes obtained from a biological sample (e.g., saliva) and exosomes obtained from a tissue (e.g., breast cancer tissue). Biomarkers within exosomes can be identical to biomarkers found in a carcinogenic tissue of a subject.

An exosome can be a cell-of-origin specific exosome. An exosome can be derived from a tumor or cancer cell. The cell-of-origin for an exosome can be, for example, lung, pancreas, stomach, intestine, bladder, kidney, ovary, testis, skin, colorectal, breast, prostate, brain, esophagus, liver, placenta, or fetal cell. In some embodiments, the cell-of-origin of an exosome is the breast tissue.

A method of the disclosure can comprise assaying biomarkers released from an exosome. In some embodiments, exosomal biomarkers can be directly assayed from the biological samples, such that one or more biomarkers of the exosomes are analyzed without prior isolation, purification, or concentration of the exosomes from the biological sample. In some embodiments, exosomes can be isolated from a biological sample and enriched prior to biomarker analysis.

Exosome can be purified or concentrated prior to analysis. Analysis of an exosome can include quantitating the amount of one or more exosome populations of a biological sample. For example, a heterogeneous population of exosomes can be quantitated, or a homogeneous population of exosomes, such as a population of exosomes with a particular biomarker profile, or derived from a particular cell type (cell-of-origin specific exosomes) can be isolated from a heterogeneous population of exosomes and quantitated. Analysis of an exosome can also include detecting, quantitatively or qualitatively, a particular biomarker profile or a bio-signature, of an exosome. An enriched population of exosomes can be obtained from a biological sample derived from any cell or cells capable of producing and releasing exosomes into the bodily fluid.

Exosomes may be concentrated or isolated from a biological sample using, for example, size exclusion chromatography, density gradient centrifugation, differential centrifugation, nanomembrane ultrafiltration, immunoabsorbent capture, affinity purification, microfiuidic separation, protein purification kits, or combinations thereof.

Size exclusion chromatography, such as gel permeation columns, centrifugation or density gradient centrifugation, and filtration methods can be used for exosomal isolation. For example, exosomes can be isolated by differential centrifugation, anion exchange and/or gel permeation chromatography, sucrose density gradients, organelle electrophoresis, magnetic activated cell sorting (MACS), or with a nanomembrane ultrafiltration concentrator. Various combinations of isolation or concentration methods can be used.

Highly abundant proteins, such as albumin and immunoglobulin, may hinder isolation of exosomes from a biological sample. For example, exosomes may be isolated from a biological sample using a system that utilizes multiple antibodies that are specific to the most abundant proteins found in that biological sample. Such a system can remove up to several proteins at once, thus unveiling the lower abundance species such as cell-of-origin specific exosomes. The isolation of exosomes from a biological sample may also be enhanced by high abundant protein removal methods. The isolation of exosomes from a biological sample can be enhanced by removing serum proteins using glycopeptide capture. In addition, exosomes from a biological sample can be isolated by differential centrifugation followed by contact with antibodies directed to cytoplasmic or anti-cytoplasmic epitopes. Protein isolation kits can be used for exosomal isolation.

The presence of exosomes can be confirmed by detecting known exosomal markers such as, but not limited to MHC Class I protein, LAMP1, CD9, CD63 and CD81 via western blotting or other means of detection. Transmission Electron Microscopy (TEM), protein concentration, and Nano-Sight LM-10HS analysis can also be used to analyze the presence and purity of isolated exosomes.

Release of biomarkers from the exosomes can be carried out, for example, by lysing the exosomes. Lysis of the exosomes can be performed directly in the biological sample. Lysis of the exosomes can be performed after enrichment of the exosomal fraction. A biological sample can be subjected to lysis conditions, for example, to lyse an exosomal fraction. Lysis can be carried out for example, by sonication. Non-limiting examples of lysis methods include reagent-assisted lysis method (e.g., using detergents), reagent-less lysis methods, chemical, mechanical (e.g., using crushing, grinding, sonication), thermal (e.g., using heat), and electrical (e.g., irreversible electroporation of the lipid bilayer of the target particles).

A biological sample can be treated to remove cells (e.g., whole intact cells) prior to biomarker analysis. A sample, which is devoid of cells, can be subjected to exosome isolation and enrichment. A sample comprising exosomes can be preserved and/or stored prior to biomarker analysis.

Methods of the disclosure can employ amplification of nucleic acids. The amplified nucleic acids can be analyzed using, for example, massively parallel sequencing (e.g., next generation sequencing methods) or hybridization platforms. Suitable amplification reactions can be exponential or isothermal, and can include any DNA amplification reaction, including but not limited to PCR, strand displacement amplification (SDA), ligase chain reaction (LCR), linear amplification, multiple displacement amplification (MDA), rolling circle amplification (RCA), or a combination thereof.

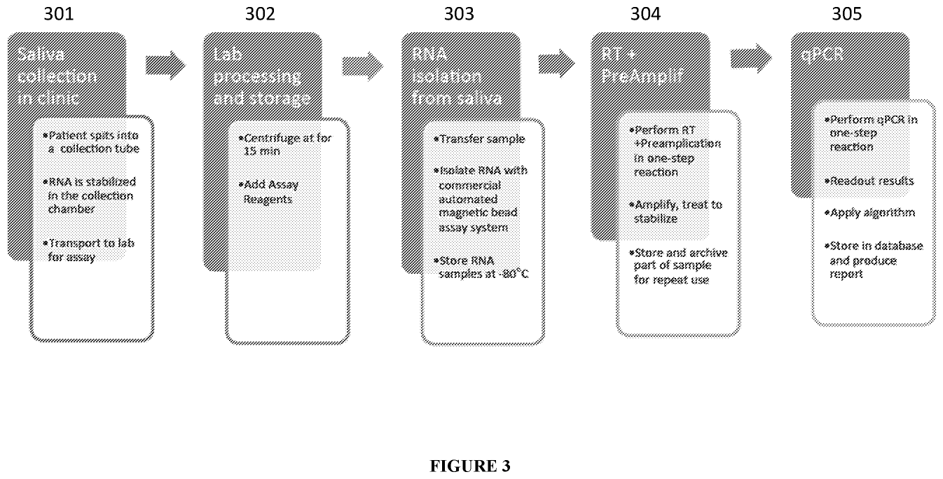

A method of the disclosure can comprise biomarker detection and analysis. Results from biomarker analysis can be used to generate a biomarker signature for a subject. FIG. 3 illustrates the workflow of an exemplary method for biomarker analysis from a saliva sample for assessing cancer in a subject. Saliva can be collected from a subject (301) by, for example, spitting into a collection tube. The saliva sample is then transported to the lab for processing and storage (302). The sample can be transported to a lab for assaying. The sample can be centrifuged. Assay reagents can be added to the sample. The exosomal fraction of the saliva sample that comprises RNA can be isolated and/or stabilized (303). RNA can be isolated from the sample with a suitable technique, for example, a magnetic bead assay system. The isolated RNA sample can be stored (e.g., at −80° C.) for later processing and analysis. In some embodiments, the isolated RNA sample can be processed further without storage. The RNA can be reverse-transcribed (e.g., using RT-PCR) to generate cDNA, and a pre-amplification step can be performed (304). In some embodiments, the RNA can be reverse-transcribed and pre-amplified in a one-step reaction. In some embodiments, reverse transcription and pre-amplification can be performed in separate steps. In some embodiments, a pre-amplification may not be performed. The cDNA can be amplified. The cDNA can be treated, for example, to increase stability. The cDNA can be stored for later processing. In some embodiments, the cDNA can be processed without storage. qPCR can be performed on the cDNA (305). In some embodiments, the qPCR can be carried out in a one-step reaction. Data from the qPCR can be analyzed to detect expression levels of candidate biomarker genes. Purification steps can be added before, after, or during any of the steps in the workflow. In some embodiments, RNA sequencing can be used for analysis of the RNA. In some embodiments, targeted RNA sequencing can be used for analysis of the RNA. In some embodiments, miRNA or small RNA sequencing can be used for analysis of the RNA.

Biomarker detection can comprise use of, for example, microarray analysis, polymerase chain reaction (PCR) including PCR-based methods such as RT-PCR and quantitative PCR (qPCR), hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), immunoblotting, immunoprecipitation, an enzyme-linked immunosorbent assay (ELISA), a radioimmunoassay (MA), flow cytometry, electron microscopy, genetic testing using G-banded karotyping, fragile X testing, chromosomal microarray (CMA, also known as comparative genomic hybridization (CGH)) (e.g., to test for submicroscopic genomic deletions and/or duplications), array-based comparative genomic hybridization, detecting single nucleotide polymorphisms (SNPs) with arrays, subtelomeric fluorescence in situ hybridization (ST-FISH) (e.g., to detect submicroscopic copy-number variants (CNVs)), expression profiling, DNA microarray, RNA microarray, mRNA microarray, miRNA microarray, high-density oligonucleotide microarray, whole-genome RNA expression array, peptide microarray, enzyme-linked immunosorbent assay (ELISA), genome sequencing, DNA sequencing, RNA sequencing, miRNA sequencing, de novo sequencing, 454 sequencing (Roche), pyrosequencing, Helicos True Single Molecule Sequencing, SOLiD™ sequencing (Applied Biosystems, Life Technologies), SOLEXA sequencing (Illumina sequencing), nanosequencing, chemical-sensitive field effect transistor (chemFET) array sequencing (Ion Torrent), ion semiconductor sequencing (Ion Torrent), DNA nanoball sequencing, nanopore sequencing, Pacific Biosciences SMRT sequencing, Genia Technologies nanopore single-molecule DNA sequencing, Oxford Nanopore single-molecule DNA sequencing, polony sequencing, copy number variation (CNV) analysis sequencing, small nucleotide polymorphism (SNP) analysis, immunohistochemistry (IHC), immunoctyochemistry (ICC), mass spectrometry, tandem mass spectrometry, matrix-assisted laser desorption ionization time of flight mass spectrometry (MALDI-TOF MS), in-situ hybridization, fluorescent in-situ hybridization (FISH), chromogenic in-situ hybridization (CISH), silver in situ hybridization (SISH), polymerase chain reaction (PCR), digital PCR (dPCR), reverse transcription PCR, quantitative PCR (Q-PCR), single marker qPCR, real-time PCR, nCounter Analysis (Nanostring technology), Western blotting, Southern blotting, SDS-PAGE, gel electrophoresis, and Northern blotting, or any combination thereof.

A method of the disclosure can comprise quantifying the expression of genes. The expression of a gene can be quantified at a transcriptomic level (e.g., RNA, mRNA, miRNA), a proteomic level (e.g., protein, polypeptide), or a combination thereof. The gene can be a cancer-related gene. The gene can be a gene in a breast cancer pathway. The gene can be an oncogene. The gene can be associated with a hallmark of cancer.

Expression analysis can be carried out on, for example, RNA extracted from exosomes. The RNA can be, for example, total RNA, mRNA, miRNA, and tRNA. In some embodiments, the exosomes can be cell-of-origin specific exosomes. Expression patterns generated from these exosomes can be indicative of a given disease state, disease stage, therapy related signature, or physiological condition. Once the total RNA has been isolated, complementary DNA (cDNA) can be generated. qRT-PCR assays for specific mRNA targets can then be performed. In some embodiments, expression microarrays can be performed to detect and identify highly multiplexed sets of expression markers. Methods for establishing gene expression profiles can include determining the amount of RNA that is produced by a gene that can code for a protein or peptide. This can be accomplished by quantitative reverse transcriptase PCR (qRT-PCR), competitive RT-PCR, real time RT-PCR, differential display RT-PCR, Northern Blot analysis, sequencing, or other tests. While it is possible to conduct these techniques using individual PCR reactions, it is also possible to amplify complementary DNA (cDNA) produced from mRNA and analyze it.

qPCR or real-time PCR can refer to PCR methods wherein an amount of detectable signal is monitored with each cycle of PCR. A cycle threshold (Ct) wherein a detectable signal reaches a detectable level can be determined. The lower the Ct value, the greater the concentration of the interrogated allele can be. Data can be collected during the exponential growth (log) phase of PCR, wherein the quantity of the PCR product is directly proportional to the amount of template nucleic acid. Systems for real-time PCR Can include the ABI 7700 and 7900HT Sequence Detection Systems. The increase in signal during the exponential phase of PCR can provide a quantitative measurement of the amount of templates containing the mutant allele.

Biomarkers can be assayed by allele-specific PCR, which can include specific primers to amplify and discriminate between two alleles of a gene simultaneously. In some embodiments, biomarkers can be assayed to detect single-strand conformation polymorphism (SSCP), which involves the electrophoretic separation of single-stranded nucleic acids based on differences in sequence and DNA and RNA aptamers. DNA and RNA aptamers can be short oligonucleotide sequences that can be selected from random pools based on their ability to bind a particular molecule with high affinity.

In some embodiments, the differential expression of a biomarker can be determined by analyzing RNA. The method can include production of corresponding cDNA, and then analyzing the resulting DNA.

In some embodiments, the method can comprise RNA sequencing. For example, the method can include one or more or the following: extraction of RNA, fragmenting, cDNA generation, sequencing library preparation, and high-throughput sequencing (e.g., next generation sequencing, massively parallel sequencing). In some embodiments, the method can comprise use of target-specific probes for a biomarker disclosed herein. In some embodiments, the method can comprise use of microarrays specific (e.g., for miRNA, mRNA).

In some embodiments, small RNA sequencing or miRNA sequencing can be used for analysis of RNA. miRNA sequencing can comprise generation of an RNA library made from RNA (e.g., obtained from saliva) containing miRNAs and other small RNAs.

Biomarker analysis can include, for example, determining absence of a mutation (e.g., wild-type) or presence of one or more mutations (e.g., a de novo mutation, nonsense mutation, missense mutation, silent mutation, frameshift mutation, insertion, substitution, point mutation, single nucleotide polymorphism (SNP), single nucleotide variant, de novo single nucleotide variant, deletion, rearrangement, amplification, chromosomal translocation, interstitial deletion, chromosomal inversion, loss of heterozygosity, loss of function, gain of function, dominant negative, or lethal); nucleic acid modification (e.g., methylation); or presence or absence of a post-translational modification on a protein (e.g., acetylation, alkylation, amidation, biotinylation, glutamylation, glycosylation, glycation, glycylation, hydroxylation, iodination, isoprenylation, lipoylation, prenylation, myristoylation, farnesylation, geranylgeranylation, ADP-ribosylation, oxdiation, palmitoylation, pegylation, phosphatidylinositol addition, phosphopantetheinylation, phosphorylation, polysialyation, pyroglutamate formation, arginylation, sulfation, or selenoylation).

The methods described herein can use one or more next-generation sequencing or high throughput sequencing such as but not limited to those methods described in U.S. Pat. Nos. 7,335,762; 7,323,305; 7,264,929; 7,244,559; 7,211,390; 7,361,488; 7,300,788; and 7,280,922.

Next-generation sequencing techniques can include, for example, Helicos True Single Molecule Sequencing (tSMS) (Harris T. D. et al. (2008) Science 320:106-109); 454 sequencing (Roche) (Margulies, M. et al. 2005, Nature, 437, 376-380); SOLiD technology (Applied Biosystems); SOLEXA sequencing (Illumina); single molecule, real-time (SMRT™) technology of Pacific Biosciences; nanopore sequencing (Soni GV and Meller A. (2007) Clin Chem 53: 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g. Oxford Nanopore, Genia Technologies, and Nabsys).

In some embodiments, the next generation sequencing technique can be 454 sequencing (Roche) (see e.g., Margulies, M et al. (2005) Nature 437: 376-380). 454 sequencing can involve two steps. In the first step, DNA can be sheared into fragments of approximately 300-800 base pairs, and the fragments can be blunt ended. Oligonucleotide adaptors can then ligated to the ends of the fragments. The adaptors can serve as sites for hybridizing primers for amplification and sequencing of the fragments. The fragments can be attached to DNA capture beads, e.g., streptavidin-coated beads using, e.g., Adaptor B, which can contain 5′-biotin tag. The fragments can be attached to DNA capture beads through hybridization. A single fragment can be captured per bead. The fragments attached to the beads can be PCR amplified within droplets of an oil-water emulsion. The result can be multiple copies of clonally amplified DNA fragments on each bead. The emulsion can be broken while the amplified fragments remain bound to their specific beads. In a second step, the beads can be captured in wells (pico-liter sized; PicoTiterPlate (PTP) device). The surface can be designed so that only one bead fits per well. The PTP device can be loaded into an instrument for sequencing. Pyrosequencing can be performed on each DNA fragment in parallel. Addition of one or more nucleotides can generate a light signal that can be recorded by a CCD camera in a sequencing instrument. The signal strength can be proportional to the number of nucleotides incorporated.

Pyrosequencing can make use of pyrophosphate (PPi) which can be released upon nucleotide addition. PPi can be converted to ATP by ATP sulfurylase in the presence of adenosine 5′ phosphosulfate. Luciferase can use ATP to convert luciferin to oxyluciferin, and this reaction can generate light that can be detected and analyzed. The 454 Sequencing system used can be GS FLX+ system or the GS Junior System.

The next generation sequencing technique can be SOLiD technology (Applied Biosystems; Life Technologies). In SOLiD sequencing, genomic DNA can be sheared into fragments, and adaptors can be attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured and beads can be enriched to separate the beads with extended templates. Templates on the selected beads can be subjected to a 3′ modification that permits bonding to a glass slide. A sequencing primer can bind to adaptor sequence. A set of four fluorescently labeled di-base probes can compete for ligation to the sequencing primer. Specificity of the di-base probe can be achieved by interrogating every first and second base in each ligation reaction. The sequence of a template can be determined by sequential hybridization and ligation of partially random oligonucleotides with a determined base (or pair of bases) that can be identified by a specific fluorophore. After a color is recorded, the ligated oligonucleotide can be cleaved and removed and the process can be then repeated. Following a series of ligation cycles, the extension product can be removed and the template can be reset with a primer complementary to the n−1 position for a second round of ligation cycles. Five rounds of primer reset can be completed for each sequence tag. Through the primer reset process, most of the bases can be interrogated in two independent ligation reactions by two different primers. Up to 99.99% accuracy can be achieved by sequencing with an additional primer using a multi-base encoding scheme.

The next generation sequencing technique can be SOLEXA sequencing (ILLUMINA sequencing). ILLUMINA sequencing can be based on the amplification of DNA on a solid surface using fold-back PCR and anchored primers. ILLUMINA sequencing can involve a library preparation step. Genomic DNA can be fragmented, and sheared ends can be repaired and adenylated. Adaptors can be added to the 5′ and 3′ ends of the fragments. The fragments can be size selected and purified. ILLUMINA sequence can comprise a cluster generation step. DNA fragments can be attached to the surface of flow cell channels by hybridizing to a lawn of oligonucleotides attached to the surface of the flow cell channel. The fragments can be extended and clonally amplified through bridge amplification to generate unique clusters. The fragments become double stranded, and the double stranded molecules can be denatured. Multiple cycles of the solid-phase amplification followed by denaturation can create several million clusters of approximately 1,000 copies of single-stranded DNA molecules of the same template in each channel of the flow cell. Reverse strands can be cleaved and washed away. Ends can be blocked, and primers can by hybridized to DNA templates. ILLUMINA sequencing can comprise a sequencing step. Hundreds of millions of clusters can be sequenced simultaneously. Primers, DNA polymerase and four fluorophore-labeled, reversibly terminating nucleotides can be used to perform sequential sequencing. All four bases can compete with each other for the template. After nucleotide incorporation, a laser can be used to excite the fluorophores, and an image is captured and the identity of the first base is recorded. The 3′ terminators and fluorophores from each incorporated base are removed and the incorporation, detection and identification steps are repeated. A single base can be read each cycle. In some embodiments, a HiSeq system (e.g., HiSeq 2500, HiSeq 1500, HiSeq 2000, or HiSeq 1000) is used for sequencing. In some embodiments, a MiSeq personal sequencer is used. In some embodiments, a Genome Analyzer IIx is used.

The next generation sequencing technique can comprise real-time (SMRT™) technology by Pacific Biosciences. In SMRT, each of four DNA bases can be attached to one of four different fluorescent dyes. These dyes can be phospholinked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zeptoliters (10˜21 liters) can be created. The tiny detection volume can provide 1000-fold improvement in the reduction of background noise. Detection of the corresponding fluorescence of the dye can indicate which base was incorporated. The process can be repeated.

The next generation sequencing method can comprise nanopore sequencing (See e.g., Soni GV and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridlON system. A single nanopore can be inserted in a polymer membrane across the top of a microwell. Each microwell can have an electrode for individual sensing. The microwells can be fabricated into an array chip, with 100,000 or more microwells (e.g., more than about 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or S102). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi:10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys. Genomic DNA can be fragmented into strands of average length of about 100 kb. The 1OO kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM/Roche. A electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing method can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some embodiments, an IONPROTON™ Sequencer is used to sequence nucleic acid. In some embodiments, an IONPGM™ Sequencer is used.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Ad1) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Ad1 adapter. A restriction enzyme (e.g., Acul) can be applied, and the DNA can be cleaved 13 bp to the left of the Ad1 to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template. Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

The next generation sequencing technique can be Helicos True Single Molecule Sequencing (tSMS) (see e.g., Harris T. D. et al. (2008) Science 320:106-109). In the tSMS technique, a DNA sample can be cleaved into strands of approximately 100 to 200 nucleotides, and a polyA sequence can be added to the 3′ end of each DNA strand. Each strand can be labeled by the addition of a fluorescently labeled adenosine nucleotide. The DNA strands can then be hybridized to a flow cell, which can contain millions of oligo-T capture sites immobilized to the flow cell surface. The templates can be at a density of about 100 million templates/cm2. The flow cell can then be loaded into an instrument, e.g., HELISCOPE™ sequencer, and a laser can illuminate the surface of the flow cell, revealing the position of each template. A CCD camera can map the position of the templates on the flow cell surface. The template fluorescent label can then be cleaved and washed away. The sequencing reaction can begin by introducing a DNA polymerase and a fluorescently labeled nucleotide. The oligo-T nucleic acid can serve as a primer. The DNA polymerase can incorporate the labeled nucleotides to the primer in a template directed manner. The DNA polymerase and unincorporated nucleotides can be removed. The templates that have directed incorporation of the fluorescently labeled nucleotide can be detected by imaging the flow cell surface. After imaging, a cleavage step can remove the fluorescent label, and the process can be repeated with other fluorescently labeled nucleotides until a desired read length is achieved. Sequence information can be collected with each nucleotide addition step. The sequencing can be asynchronous. The sequencing can comprise at least 1 billion bases per day or per hour.

The sequencing technique can comprise paired-end sequencing in which both the forward and reverse template strand can be sequenced. In some embodiments, the sequencing technique can comprise mate pair library sequencing. In mate pair library sequencing, DNA can be fragments, and 2-5 kb fragments can be end-repaired (e.g., with biotin labeled dNTPs). The DNA fragments can be circularized, and non-circularized DNA can be removed by digestion. Circular DNA can be fragmented and purified (e.g., using the biotin labels). Purified fragments can be end-repaired and ligated to sequencing adaptors.

A sequence read can be about, more than about, less than about, or at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, or 3000 bases. In some embodiments, a sequence read is about 10 to about 50 bases, about 10 to about 100 bases, about 10 to about 200 bases, about 10 to about 300 bases, about 10 to about 400 bases, about 10 to about 500 bases, about 10 to about 600 bases, about 10 to about 700 bases, about 10 to about 800 bases, about 10 to about 900 bases, about 10 to about 1000 bases, about 10 to about 1500 bases, about 10 to about 2000 bases, about 50 to about 100 bases, about 50 to about 150 bases, about 50 to about 200 bases, about 50 to about 500 bases, about 50 to about 1000 bases, about 100 to about 200 bases, about 100 to about 300 bases, about 100 to about 400 bases, about 100 to about 500 bases, about 100 to about 600 bases, about 100 to about 700 bases, about 100 to about 800 bases, about 100 to about 900 bases, or about 100 to about 1000 bases.

The number of sequence reads from a sample can be about, more than about, less than about, or at least about 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The depth of sequencing of a sample can be about, more than about, less than about, or at least about 1×, 2×, 3×, 4×, 5×, 6×, 7×, 8×, 9×, 1O×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 1OO×, HO×, 120×, 130×, 140×, 150×, 160×, 170×, 180×, 190×, 200×, 300×, 400×, 500×, 600×, 700×, 800×, 900×, 1OOO×, 1500×, 2000×, 2500×, 3000×, 3500×, 4000×, 4500×, 5000×, 5500×, 6000×, 6500×, 7000×, 7500×, 8000×, 8500×, 9000×, 9500×, or 10,000×. The depth of sequencing of a sample can about 1× to about 5×, about 1× to about 1O×, about 1× to about 20×, about 5× to about 10×, about 5× to about 20×, about 5× to about 3 O×, about 1O× to about 20×, about 1O× to about 25×, about 1O× to about 3 O×, about 1O× to about 40×, about 3O× to about 1OO×, about 1OO× to about 200×, about 1OO× to about 500×, about 500× to about 1OOO×, about 1OOO×, to about 2000×, about 1OOO× to about 5000×, or about 5000× to about IO,OOO_{χ}. Depth of sequencing can be the number of times a sequence (e.g., a genome) is sequenced. In some embodiments, the Lander/Waterman equation is used for computing coverage. The general equation can be: C=LN/G, where C=coverage; G=haploid genome length; L=read length; and N=number of reads.

In some embodiments, different barcodes can be added to polynucleotides in different samples (e.g., by using primers or adaptors), and the different samples can be pooled and analyzed in a multiplexed assay. The barcode can allow the determination of the sample from which a polynucleotide originated.

In some embodiments, a method can comprise use of biomarker analysis and an additional screening test for a health condition. In some embodiments, a method can comprise performing biomarker analysis on a subject with an ambiguous, positive, or negative result from an additional screening test. The additional screening test can be a prescreening test for a health condition. The additional screening test can be a test that evaluates the risk of a subject for developing a health condition. The additional screening method can be performed before, after, or in conjunction with biomarker analysis. Such a combinatorial approach comprising two or more screening methods can increase accuracy, sensitivity, and/or specificity of detection. Additionally, a combinatorial method can be useful for increasing early cancer detection, guiding additional screening options for subjects at high risk or with dense breast tissue and/or ambiguous results on screenings tests such as mammograms.

Various additional screening tests or methods are suitable for use with a method of the disclosure. Non-limiting examples of such screening tests include imaging methods (using for example, x-rays, sound waves, radioactive particles, or magnetic fields), mammography, scintimammography, breast exams (e.g., clinical and self), genetic screening (e.g., BRCA testing), ultrasound, magnetic resonance imaging (MRI), molecular breast imaging, biopsy, ultrasonography, non-invasive diagnostic method, for example, comprising quantification of circulating cell-free nucleic acid, such as DNA (e.g., cfdDNA) or RNA (e.g., cfRNA) associated with a health condition, and any combination thereof. In some embodiments, the additional screening test is mammogram.

In some embodiments, the additional method can be a biopsy. In some embodiments, the additional screening test can be genetic screening (e.g., BRCA testing). In some embodiments, the additional screening method can be a non-invasive diagnostic method, for example, comprising quantification of circulating cell-free nucleic acid, such as DNA (e.g., cfdDNA) or RNA (e.g., cfRNA) associated with a health condition. In some embodiments, circulating cell-free nucleic acids are quantified from a biofluid biological sample. In some embodiments, the sample can be, for example, blood, plasma, serum, urine, or stool. In some embodiments, quantification can be achieved through high throughput sequencing of the cell-free nucleic acid.

In some embodiments, the additional screening method is a prescreening test for breast cancer such as an imaging test, for example, mammography. For example, biomarker analysis can be used in combination with annual breast cancer screening or testing of subjects with high-risk for breast cancer performed using, for example, mammography. In some embodiments, a method of the disclosure can comprise a combination of a saliva-based biomarker assay with a mammogram, computed tomography (CT) scan, breast magnetic resonance imaging (MM) scan, or a combination thereof for breast cancer detection.

A score obtained from a mammogram can be adjusted or a new score generated using a method of the disclosure. A mammogram result can be expressed in terms of the Breast Imaging Reporting and Data system (BI-RADS) Assessment Category (i.e., BI-RADS score), which can range from 0 (Incomplete) to 6 (Known biopsy—proven malignancy). Mammograms can be scored on a scale from 1-5 (1=normal, 2=benign, 3=indeterminate, 4=suspicious of malignancy, 5=malignant). For example, a subject with a mammogram score of 3 can be reclassified as a 1 based on results from biomarker analysis.

A method comprising, for example, biomarker analysis and an additional screening test or results from an additional screening test, can increase sensitivity and/or specificity of detection compared with that obtained with the screening test alone. In some embodiments, specificity can be increased or maximized by correctly identifying a subject as a negative for a health condition. For example, by using a combinatorial method of the disclosure on “call-backs” (e.g., patients that are normal but have ambiguous mammograms) to correctly identify the subject as a negative for breast cancer. In some embodiments, sensitivity can be increased or maximized by correctly identifying a subject as a positive for a health condition. For example, by using a combinatorial method of the disclosure on a subject with a high risk of cancer or with high density breast tissue and correctly identifying the subject as a positive for breast cancer.

A method of the disclosure can comprise generating a risk score for a health condition for a subject. The risk score can be indicative of the risk of developing a health condition by the subject. A risk score can be calculated based on results of a biomarker assay. A risk score can be calculated, combined, and/or adjusted based on data from an additional screening test. A risk score can be provided in conjunction with a mammogram result, and the combined information can be used to determine, for example, the probability that a patient has cancer.

A method of the disclosure can comprise classifying subjects into two or more groups based on their biomarker signature (e.g., obtained from results of biomarker analysis) alone or in combination with results from an additional screening test. Subjects can be classified into a positive (e.g., breast cancer positive) or a negative (breast cancer negative) group for a health condition. Subjects can be classified into high risk, low risk, and intermediate risk categories for a health condition. In one example, a biomarker signature can be used to determine that a subject is at a low risk for breast cancer and may not need to undergo annual mammogram screening. In another example, a patient can be classified as having a high-risk of breast cancer based on their biomarker signature and a prescreening test, and can be recommended to increase surveillance for cancer detection.

A method of the disclosure can provide a risk that can be indicative of a current real-time state of a subject. The real-time state can be related to a given disease state, disease stage, therapy related signature, or physiological condition. Because the risk can be reflective of the current state of the subject, a method of the disclosure can be performed repeatedly over the patient's life, such as annually, semi-annually, or quarterly. For example, high-risk patients can have a method of the disclosure performed quarterly. A method of the disclosure can differ from genetic testing, which may be performed once in the subject's lifetime. A genetic test (e.g., breast cancer genetic testing such as for BRCA1 or BRCA2) can be conducted using any cell from the subject, and can represent lifetime risk. A genetic test may not be indicative of a subject's current health state, while a method of the disclosure can determine risk at the time of testing.

A method of the disclosure can have a low false-positive rate. In some embodiments, the false-positive rate for the methods of the disclosure can be, for example, less than about 1%, less than about 2%, less than about 3%, less than about 4%, less than about 5%, less than about 6%, about 7%, less than about 8%, less than about 9%, less than about 10%, less than about 11%, less than about 12%, less than about 13%, less than about 14%, less than about 15%, less than about 16%, less than about 17%, less than about 18%, less than about 19%, or less than about 20%.

The sensitivity of a method of the disclosure can be, for example, about 75%, about 80%, about 83%, about 85%, about 87%, about 90%, about 91%, about 92%, about 93%, about 94%, about 95%, about 96%, about 97%, about 98%, about 99%, about 99.5%, or 100%. The sensitivity of methods of the disclosure can be, for example, at least 75%, at least 80%, at least 83%, at least 85%, at least 87%, at least 90%, at least 93%, at least 95%, at least 96%, at least 97%, at least 98%, at least 99%, or at least 99.5%. The sensitivity of methods of the disclosure can be, for example, greater than 75%, greater than 80%, greater than 83%, greater than 85%, greater than 87%, greater than 90%, greater than 93%, greater than 95%, greater than 96%, greater than 97%, greater than 98%, greater than 99%, or greater than 99.5%. In some embodiments, the sensitivity of the methods of the disclosure is about 83%. In some embodiments, the sensitivity of the methods of the disclosure is greater than 83%.

The specificity of a method of the disclosure can be, for example, about 75%, about 80%, about 83%, about 85%, about 87%, about 90%, about 91%, about 92%, about 93%, about 94%, about 95%, about 96%, about 97%, about 98%, about 99%, about 99.5%, or 100%. The specificity of methods of the disclosure can be, for example, at least 75%, at least 80%, at least 83%, at least 85%, at least 87%, at least 90%, at least 93%, at least 95%, at least 96%, at least 97%, at least 98%, at least 99%, or at least 99.5%. The specificity of methods of the disclosure can be, for example, greater than 75%, greater than 80%, greater than 83%, greater than 85%, greater than 87%, greater than 90%, greater than 93%, greater than 95%, greater than 96%, greater than 97%, greater than 98%, greater than 99%, or greater than 99.5%. In some embodiments, the specificity of the methods of the disclosure is about 97%. In some embodiments, the specificity of the methods of the disclosure is greater than 97%.

The accuracy of a method of the disclosure can be, for example, about 75%, about 80%, about 83%, about 85%, about 87%, about 90%, about 91%, about 92%, about 93%, about 94%, about 95%, about 96%, about 97%, about 98%, about 99%, about 99.5%, or 100%. The accuracy of methods of the disclosure can be, for example, at least 75%, at least 80%, at least 83%, at least 85%, at least 87%, at least 90%, at least 93%, at least 95%, at least 96%, at least 97%, at least 98%, at least 99%, or at least 99.5%. The accuracy of methods of the disclosure can be, for example, greater than 75%, greater than 80%, greater than 83%, greater than 85%, greater than 87%, greater than 90%, greater than 93%, greater than 95%, greater than 96%, greater than 97%, greater than 98%, greater than 99%, or greater than 99.5%. In some embodiments, the accuracy of the methods of the disclosure is about 90%. In some embodiments, the accuracy of the methods of the disclosure is greater than 97%.

In some embodiments, the set of genes combined give a specificity or sensitivity of greater than 70%, 75%, 80%, 85%, 86%, 87%, 88%, 89%, 90%, 91%, 92%, 93%, 94%, 95%, 96%, 97%, 98%, 99%, or 99.5%, and/or an accuracy of at least 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% or more.

A method of the disclosure can have a high signal-to-noise ratio, which can be helpful for differentiating tumor profiles.

Subjects can be humans, patient, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, male, female, elderly adults, adults, adolescents, pre-adolescents, children, toddlers, infants.

A subject can be, for example, from 10 to 90 years of age. A subject can be, for example, 10 to 60, 18 to 25, 18 to 30, 18 to 35, 18 to 40, 18 to 45, 18 to 50, 18 to 55, 18 to 60, 18 to 65, 18 to 70, 20 to 25, 20 to 30, 20 to 35, 20 to 40, 20 to 45, 20 to 50, 20 to 55, 20 to 60, 20 to 65, 20 to 70, 25 to 30, 25 to 35, 25 to 40, 25 to 45, 25 to 50, 25 to 55, 25 to 60, 25 to 65, 25 to 70, 30 to 35, 30 to 40, 30 to 45, 30 to 50, 30 to 55, 30 to 60, 30 to 65, 30 to 70, 35 to 40, 35 to 45, 35 to 50, 35 to 55, 35 to 60, 35 to 65, 35 to 70, 40 to 45, 40 to 50, 40 to 55, 40 to 60, 40 to 65, 40 to 70, 45 to 50, 45 to 55, 45 to 60, 45 to 65, 45 to 70, 50 to 55, 50 to 60, 50 to 65, 50 to 70, 55 to 60, 55 to 65, 55 to 70, 60 to 65, 60 to 70, or 65 to 70 years of age. In some embodiments, the subject can be between 18 to 40 years of age. In some embodiments, the subject can be less than 40 years of age. In some embodiments, the subject can be less than 35 years of age. In some embodiments, the subject can be less than 50 years of age. In some embodiments, the subject can be less than 60 years of age. In some embodiments, the subject can be less than 70 years of age.

The subject can have a pre-existing disease or condition, such as cancer. Alternatively, the subject may not have any known pre-existing condition. The subject may also be non-responsive to an existing or past treatment, such as a treatment for cancer. The subject may be undergoing a treatment for cancer, for example, chemotherapy.

In some embodiments, the subject can have high-density breast tissue or dense breast tissue. In some embodiments, the subject can be a high-risk subject, for example, a BRCA1 and/or a BRCA2 carrier. A subject can have a positive, negative, or ambiguous result from a prescreening test for a health condition. A subject can have a positive, negative, or ambiguous mammogram result. A subject can have an ambiguous mammogram result and dense breast tissue.

The breast density of a subject as classified by a Breast Imaging Reporting and Database Systems or BI-RADS can be Mostly fatty, Scattered density, Consistent density, or Extremely dense. Mostly fatty classification can be indicative of breasts that are made up of mostly fat and contain little fibrous and glandular tissue. This can mean the mammogram may show anything that was abnormal. Scattered density classification can be indicative of breasts that have quite a bit of fat, but there are a few areas of fibrous and glandular tissue. Consistent density classification can be indicative of breasts that have many areas of fibrous and glandular tissue that are evenly distributed through the breasts. This can make it hard to see small masses in the breast. Extremely dense category can be indicative of breasts that have a lot of fibrous and glandular tissue. This may make it hard to see a cancer on a mammogram because the cancer can blend in with the normal tissue. In some embodiments, the subject can have extremely dense breasts.

A combination of subject's data (e.g., related to age, gender, race, physical condition, breast cancer type or stage, and breast tissue density) can be used with the methods of the disclosure.

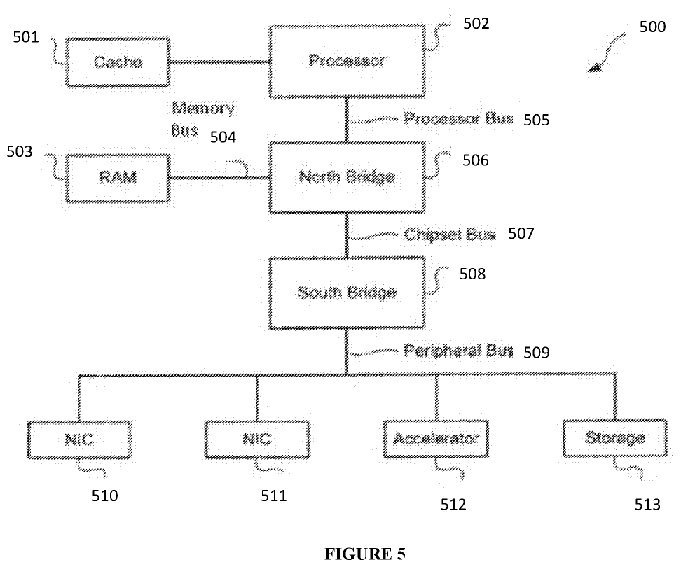

Various computer architectures are suitable for use with the disclosure. FIG. 5 is a block diagram that illustrates an example of a computer architecture system (500). The computer system 500 can be used in connection with example embodiments of the present disclosure. As depicted in FIG. 5, the example computer system can include a processor (502) for processing instructions. Non-limiting examples of processors include: Intel Core i7™, Intel Core i5™, Intel Core i3™, Intel Xeon™, AMD Opteron™, Samsung 32-bit RISC ARM 1176JZ(F)-S v1.0™ ARM Cortex-A8 Samsung S5PC100™, ARM Cortex-A8 Apple A4™, Marvell PXA 930™, or functionally-equivalent processors. Multiple threads of execution can be used for parallel processing. In some embodiments, multiple processors or processors with multiple cores can be used. In some embodiments, multiple processors or processors with multiple cores can be used in a single computer system, in a cluster, or distributed across systems over a network. In some embodiments, the multiple processors or processors with multiple cores can be distributed across systems over a network comprising a plurality of computers, cell phones, and/or personal data assistant devices.

a. Data Acquisition, Processing, and Storage

A high speed cache (501) can be connected to, or incorporated in, the processor (502) to provide high speed memory for instructions or data that have been recently, or are frequently, used by the processor (502). The processor (502) is connected to a north bridge (506) by a processor bus (505). The north bridge (506) is connected to random access memory (RAM) (503) by a memory bus (504) and manages access to the RAM (503) by the processor (502). The north bridge (506) is also connected to a south bridge (508) by a chipset bus (507). The south bridge (508) is, in turn, connected to a peripheral bus (509). The peripheral bus can be, for example, PCI, PCI-X, PCI Express, or another peripheral bus. The north bridge and south bridge, often referred to as a processor chipset, manage data transfer between the processor, RAM, and peripheral components on the peripheral bus (509). In some computer architecture systems, the functionality of the north bridge can be incorporated into the processor instead of using a separate north bridge chip.

In some embodiments, the computer architecture system (500) can include an accelerator card (512). In some embodiments, the computer architecture system (500) can include an accelerator card that is attached to the peripheral bus (509). In some embodiments, the accelerator card (512) can include field programmable gate arrays (FPGAs) or other hardware for accelerating processing.

b. Software Interface(s)

Software and data are stored in an external storage module (513) and can be loaded into the RAM (503) and/or cache (501) for use by the processor. The computer architecture system (2300) can include an operating system for managing system resources. Non-limiting examples of operating systems include: Linux, Windows™, MACOS™, BlackBerry OS™, iOS™, and other functionally-equivalent operating systems. In some embodiments, the operating system can be application software running on top of an operating system.

In FIG. 5, the computer architecture system (500) also includes network interface cards (NICs) (510 and 511) that are connected to the peripheral bus to provide network interfaces to external storage. In some embodiments, the network interface card is a Network Attached Storage (NAS) device or another computer system that can be used for distributed parallel processing.

c. Computer Networks

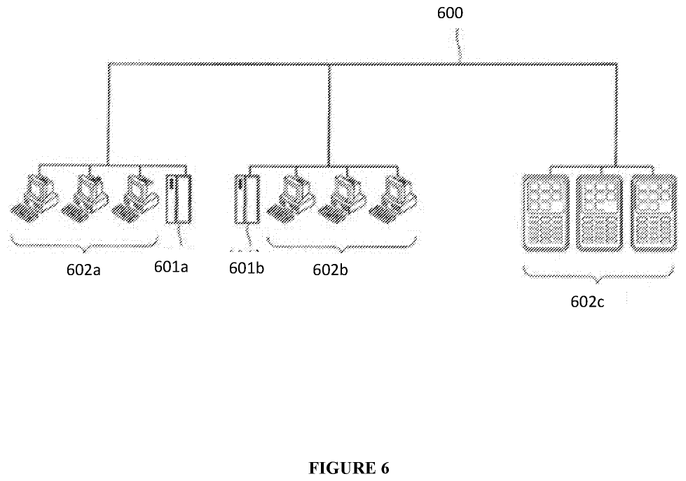

FIG. 6 is a diagram showing a computer network (600) with a plurality of computer systems (602a and 602b), a plurality of cell phones and personal data assistants (602c), and NAS devices (601a and 601b). In some embodiments, systems 602a, 602b, and 602c can manage data storage and optimize data access for data stored on NAS devices (601a and 602b). A mathematical model can be used to evaluate data using distributed parallel processing across computer systems (602a and 602b) and cell phone and personal data assistant systems (602c). Computer systems (602a and 602b) and cell phone and personal data assistant systems (602c) can also provide parallel processing for adaptive data restructuring of data stored on NAS devices (601a and 601b).

FIG. 6 illustrates an example only, and a wide variety of other computer architectures and systems can be used in conjunction with the various embodiments of the present disclosure. For example, a blade server can be used to provide parallel processing. Processor blades can be connected through a back plane to provide parallel processing. Storage can also be connected to the back plane or a NAS device through a separate network interface.

In some embodiments, processors can maintain separate memory spaces and transmit data through network interfaces, back plane, or other connectors for parallel processing by other processors. In some embodiments, some or all of the processors can use a shared virtual address memory space.

d. Virtual Systems

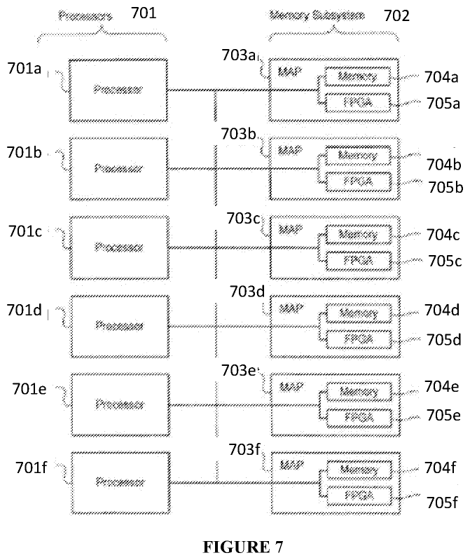

FIG. 7 is a block diagram of a multiprocessor computer system using a shared virtual address memory space. The system includes a plurality of processors (701a-701f) that can access a shared memory subsystem (702). The system incorporates a plurality of programmable hardware memory algorithm processors (MAPs) (703a-703f) in the memory subsystem (702). Each MAP (703a-703f) can comprise a memory card (704a-704f) and one or more field programmable gate arrays (FPGAs) (705a-705f). The MAPs provide configurable functional units. Algorithms or portions of algorithms can be provided to the FPGAs (705a-705f) for processing in close coordination with a respective processor. In some embodiments, each MAP is globally accessible by all of the processors. In some embodiments, each MAP can use Direct Memory Access (DMA) to access an associated memory card (704a-704f), allowing it to execute tasks independently of, and asynchronously from, the respective microprocessor (701a-701f). In some this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments. In some embodiments, the systems of the disclosure can use any combination of general processors, co-processors, FPGAs and other programmable logic devices, system on chips (SOCs), application specific integrated circuits (ASICs), and other processing and logic elements. Any variety of data storage media can be used in connection with example embodiments, including RAM, hard drives, flash memory, tape drives, disk arrays, NAS devices, and other local or distributed data storage devices and systems.

In some embodiments, the computer system can be implemented using software modules executed on any of the computer architectures and systems descried above. In some embodiments, the functions of the system can be implemented partially or completely in firmware or programmable logic devices (e.g., FPGAs) as referenced in FIG. 7, system on chips (SOCs), application specific integrated circuits (ASICs), or other processing and logic elements. For example, the Set Processor and Optimizer can be implemented with hardware acceleration through the use of a hardware accelerator card, such as an accelerator card (512) illustrated in FIG. 5.

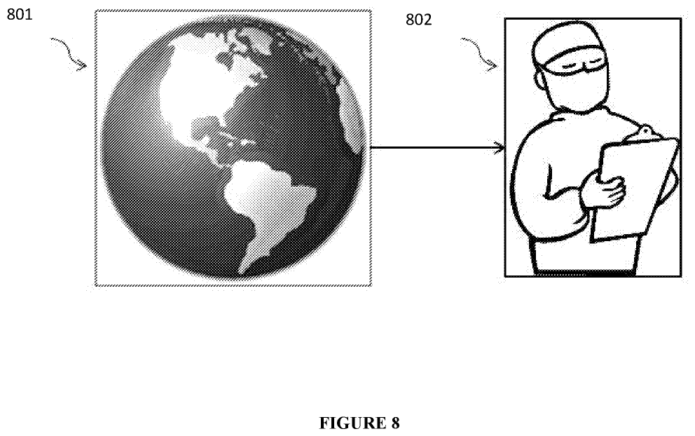

Any embodiment of the disclosure described herein can be, for example, produced and transmitted by a user within the same geographical location. A product of the disclosure can be, for example, produced and/or transmitted from a geographic location in one country and a user of the disclosure can be present in a different country. In some embodiments, the data accessed by a system of the disclosure is a computer program product that can be transmitted from one of a plurality of geographic locations (801) to a user (802). FIG. 8 illustrates a computer program product that is transmitted from a geographic location to a user. Data generated by a computer program product of the disclosure can be transmitted back and forth among a plurality of geographic locations. In some embodiments, data generated by a computer program product of the disclosure can be transmitted by a network connection, a secure network connection, an insecure network connection, an internet connection, or an intranet connection. In some embodiments, a system herein is encoded on a physical and tangible product.

### EXAMPLES

The invention is further described in detail by reference to the following examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Identification of Biomarkers

Publically available data was used to analyze gene co-expression networks. Eighteen possible biomarkers were discovered for a number of conditions. Biomarkers were discovered for conditions including breast cancer, colon cancer, lung cancer, neurodegenerative diseases, and inflammatory disorders.

### Example 2: Analysis of Gene Expression Levels

Gene expression levels were analyzed using saliva samples obtained from 10 breast cancer subjects and 10 matched and healthy controls. This study provided proof-of-concept for saliva-based breast cancer detection using microarray data from the discovery dataset of 10 patients and 10 control samples. The samples included a mixed population of, for example, races, BRCA and non-BRCA, dense samples, and non-dense samples.

A biomarker discovery phase analysis study identified about 8800 genes of relevance that could be used to determine the average connectivity between modules on a microarray (e.g., Affymetrix HG-U133 Plus 2.0 gene chip). The average connectivity of modules derived from these genes was examined to determine if the average connectivity yielded a biomarker signature with high sensitivity, high specificity, and high statistical significance. The study produced a result comprising an accuracy of about 90%.

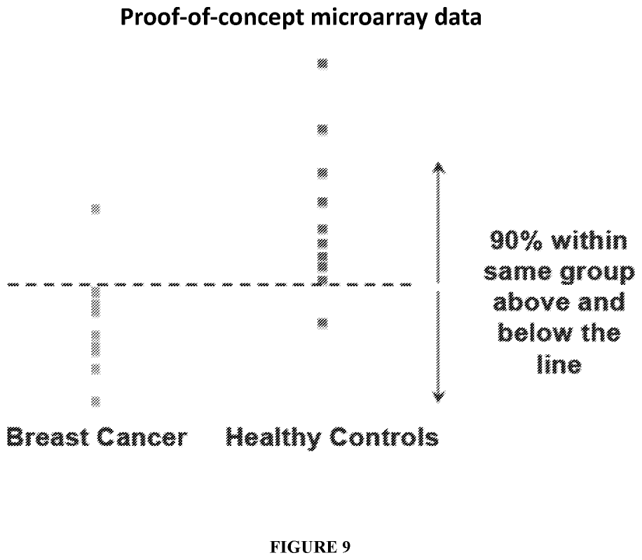

FIG. 9 illustrates the average connectivity values derived from 10 breast cancer subjects and 10 matched and healthy controls. The gene expression microarray data from which these values were derived were obtained from the NCBI Gene Expression Omnibus, GSE 20266. A comparison between the breast cancer subjects and the control subjects by t-test yielded a p-value of about 0.002. The dashed line between the two groups separated the subjects with about 90% accuracy in both directions.

The gene expression modules that contributed most to the result were then examined, and individual genes with the gene expression modules that were of greatest significance were analyzed. The analysis of individual genes with the gene expression modules that were of greatest significance was conducted to produce the most efficient subnetwork creating the separation between the control arm and the breast cancer arm in the study. The most efficient subnetwork included 4 modules containing 9 important genes, as shown in FIG. 4. FIG. 4 illustrates the principle sub-network involved in creating the separation of average connectivity derived from the microarray data that identified about 8800 genes. Module 1 (401) included SLC25A51, which can also be known as MCART1, and LCE2B; Module 2 (402) included HIST1H4K and ABCA2; Module 3 (403) included TNFRSF10A, AK092120, and DTYMK; and Module 4 (404) included Hs.161434 and ALKBH1. In an illustrative example, a “9-gene biomarker assay” or “9-gene biomarker panel” can comprise one or more of the biomarker genes identified in this example and illustrated in FIG. 4.

Correlations within this subnetwork can be reflective of the phenotypic differences to a higher degree than looking at the network as a whole. With the biomarker panel reduced to nine genes, gene expression can be examined using, for example, qPCR. Gene expression detection with, for example, qPCR, can be cheaper and more scalable than, for example, using microarrays (e.g., Affymetrix gene chips).

### Example 3: Validation of Identified Biomarkers

Initial validation of the biomarkers identified in EXAMPLE 2 was carried out on 60 patient samples for the 9-gene biomarker panel (e.g., genes in FIG. 4). The samples included a mixed population of, for example, races, BRCA and non-BRCA, dense samples, and non-dense samples. When there were complications with the mammogram, such as the presence of dense breast tissue, the data from the 9-gene assay were used to direct the need for further screening, greatly increasing detection rates.

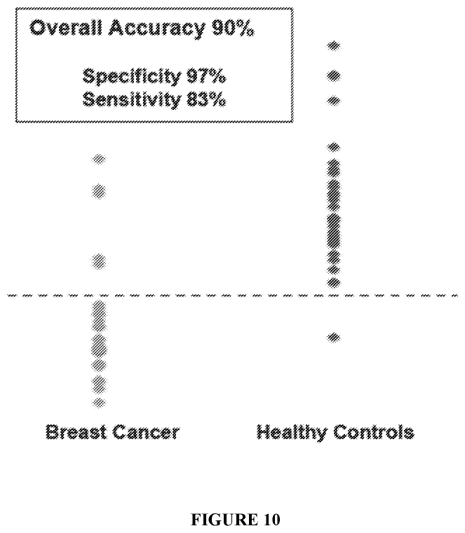

FIG. 10 illustrates scores obtained from a 9-gene assay performed using qPCR taken from a validation study of 60 subjects. The validation study included 30 breast cancer subjects identified with invasive ductal carcinoma (IDC) and 30 healthy control subjects. The results shown in FIG. 10 validated the methods used in EXAMPLE 2. The assay had a sensitivity of about 83%. The assay had a specificity of about 97% compared with a specificity level of 90% from mammograms.

The data from the 60 patient study indicated that a 9-Gene Assay for saliva-based breast cancer detection had an overall accuracy of about 90% with a sensitivity of about 83% and a specificity of about 97%. Based on these results, the 9-Gene Assay was able to detect about 83% of all women with cancer.

Results of this initial validation study showed that biomarker values transcended technology platforms (e.g., qRT-PCR and microarrays). The detection showed sensitivity and specificity levels within the range of diagnostic tests.

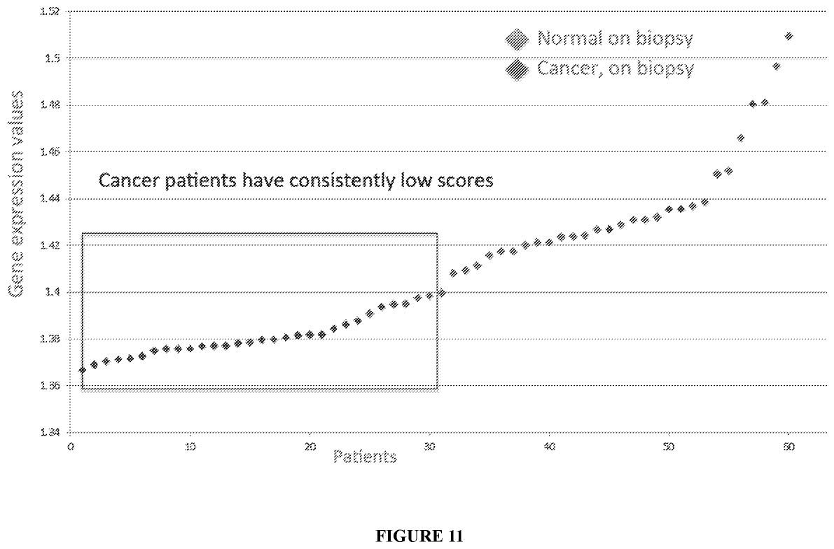

FIG. 11 illustrates serially-ordered composite gene expression values. The data demonstrate excellent separation of breast cancer subjects from control subjects (30 patient replication set). The data was obtained from a 60 patient cohort, serially ordered for the 9-gene biomarker assay.

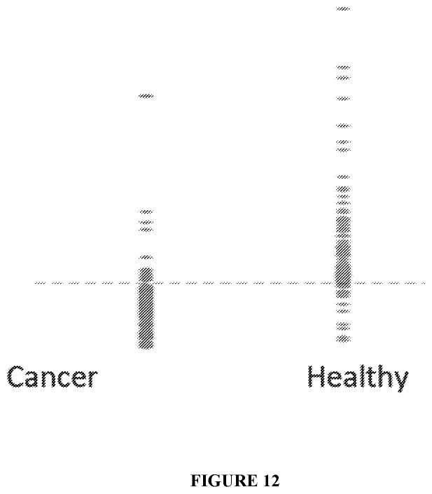

A secondary validation study was carried out on a large cohort group including 120 patients and 120 controls. The samples included a mixed population of, for example, races, BRCA-positive subjects, BRCA-negative subjects, dense samples, and non-dense samples. Data from this study are shown in FIGS. 12-25 for each of the 9 biomarker genes and 2 housekeeping genes.

FIG. 12 illustrates results of a secondary validation study for biomarker gene 5. The data were obtained from a large cohort study including 120 patient and 120 control samples. The results showed a significant separation of cancer patients and control patients. Similar results were obtained for 5 of the 9 biomarker genes from the 9-gene biomarker panel.

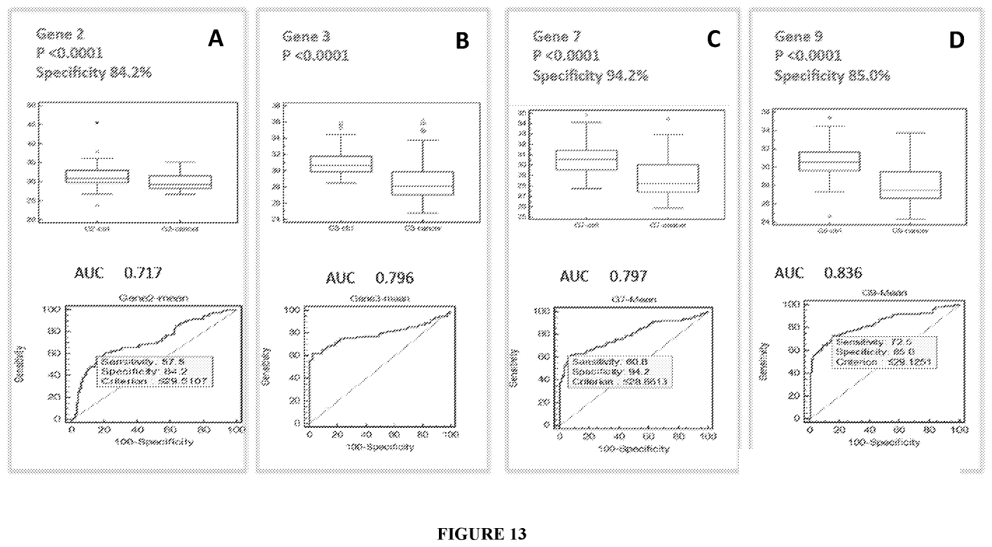

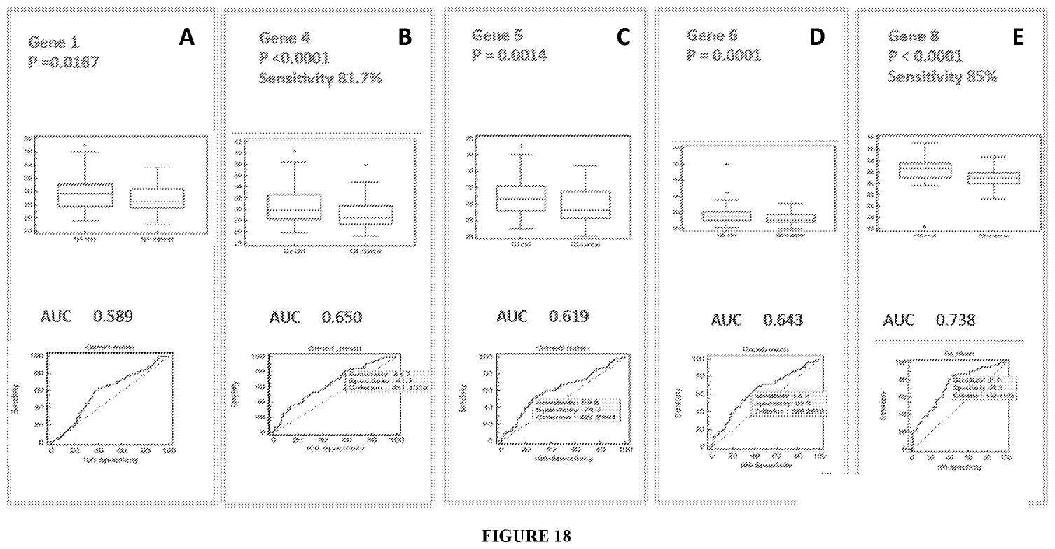

FIGS. 13A-D to FIG. 18 illustrate results of a RT-qPCR-based secondary validation study for the 9 illustrative biomarker genes. The data were obtained from a large cohort study including 120 patient and 120 control samples.

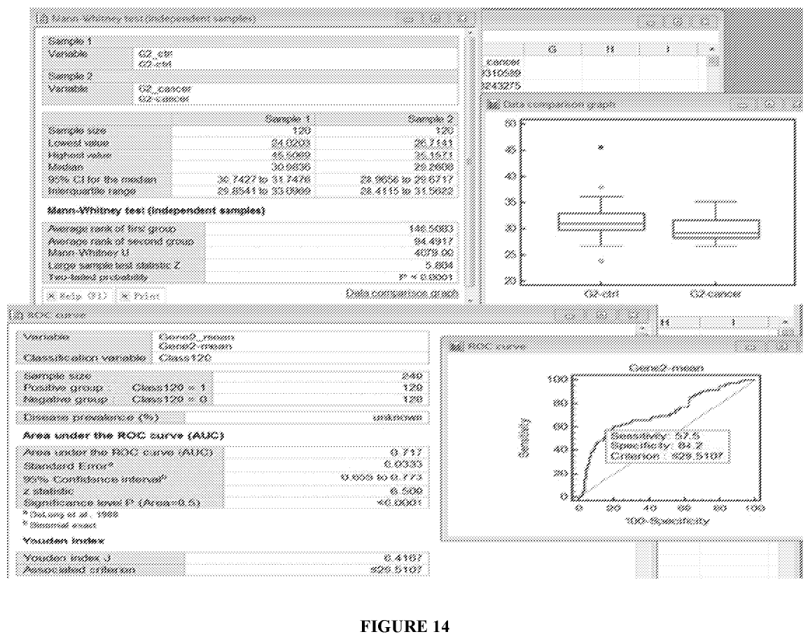

FIG. 13A shows the results of a RT-qPCR-based secondary validation study for Gene 2. Gene 2 was one of the largest genetic contributors for breast cancer in saliva samples. The data showed good separation of cancer patients from control patients, and exhibited a specificity of 84.2% with a p-value that was less than 0.0001. FIG. 14 shows parameters and results of the biomarker validation study for Gene 2.

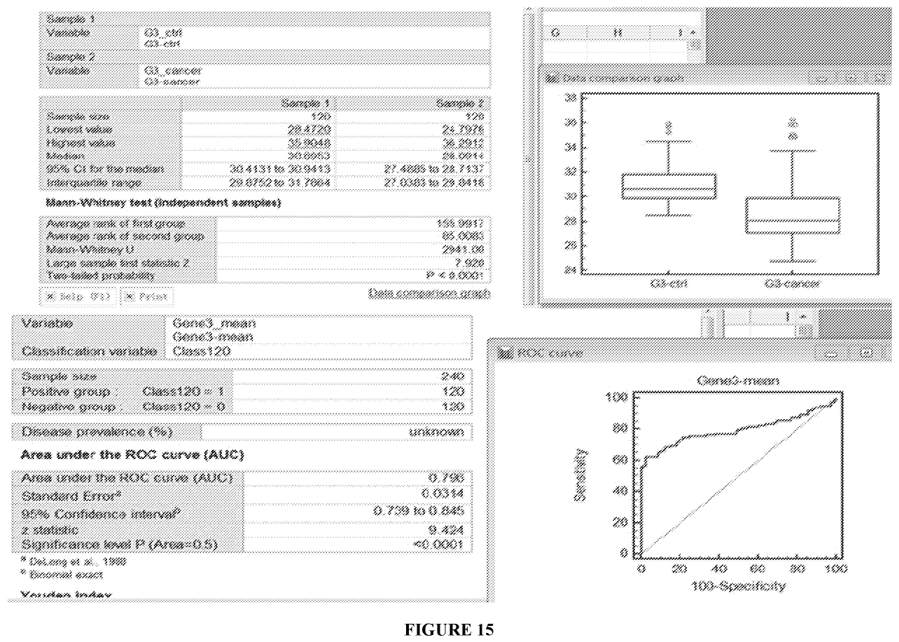

FIG. 13B shows the results of a RT-qPCR-based secondary validation study for Gene 3. Gene 3 was one of the largest genetic contributors for breast cancer in saliva samples. The data showed good separation of cancer patients from control patients, and exhibited a p-value that was less than 0.0001. FIG. 15 shows parameters and results of the biomarker validation study for Gene 3.

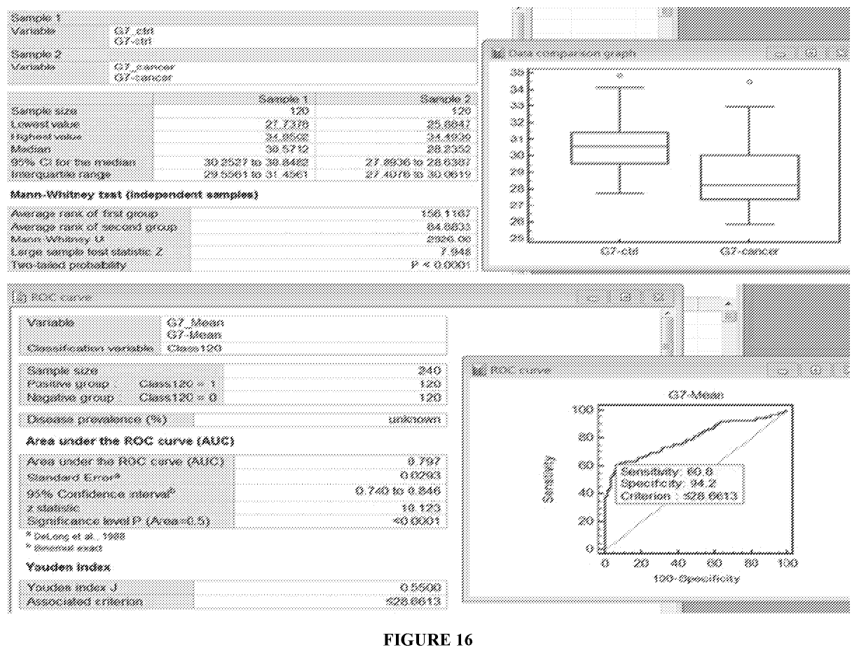

FIG. 13C shows the results of a RT-qPCR-based secondary validation study for Gene 7. Gene 7 was one of the largest genetic contributors for breast cancer in saliva samples. The data showed good separation of cancer patients from control patients, and exhibited a sensitivity of 60.8%, specificity of 94.2%, and a p-value that was less than 0.0001. FIG. 16 shows parameters and results of the biomarker validation study for Gene 7.

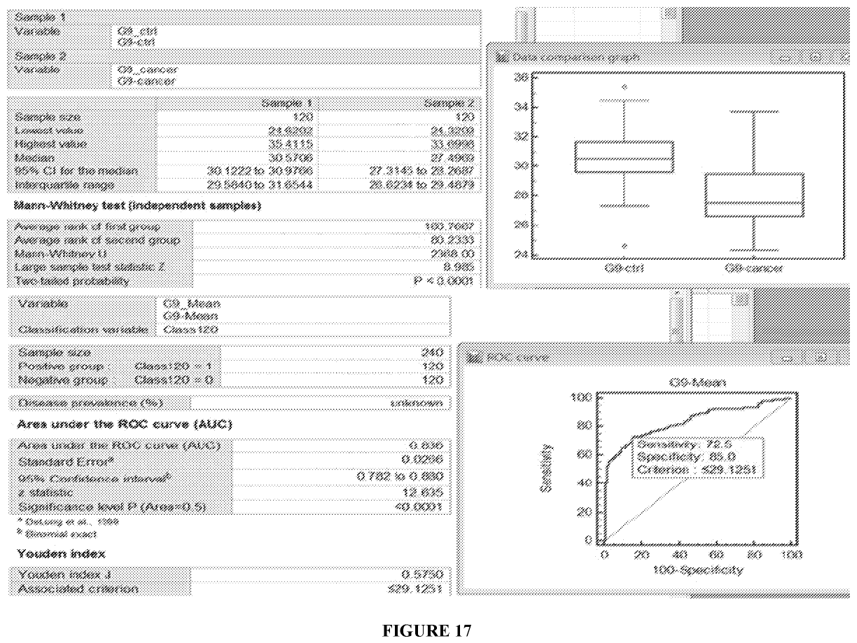

FIG. 13D shows the results of a RT-qPCR-based secondary validation study for Gene 9. Gene 9 was one of the largest genetic contributors for breast cancer in saliva samples. The data showed good separation of cancer patients from control patients, and exhibited a sensitivity of 72.5%, specificity of 85%, and a p-value that was less than 0.0001. FIG. 17 shows parameters and results of the biomarker validation study for Gene 9.

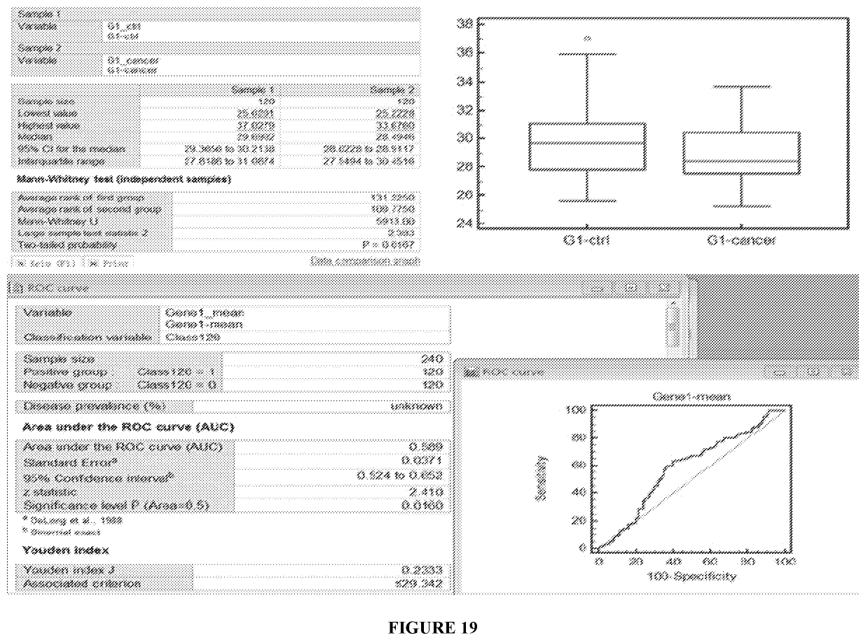

FIG. 18A shows the results of a RT-qPCR-based secondary validation study for Gene 1. Gene 1 was not one of the largest genetic contributors for breast cancer in saliva samples. The data showed good separation of cancer patients from control patients, and had a p-value of 0.0167. FIG. 19 shows parameters and results of the biomarker validation study for Gene 1.

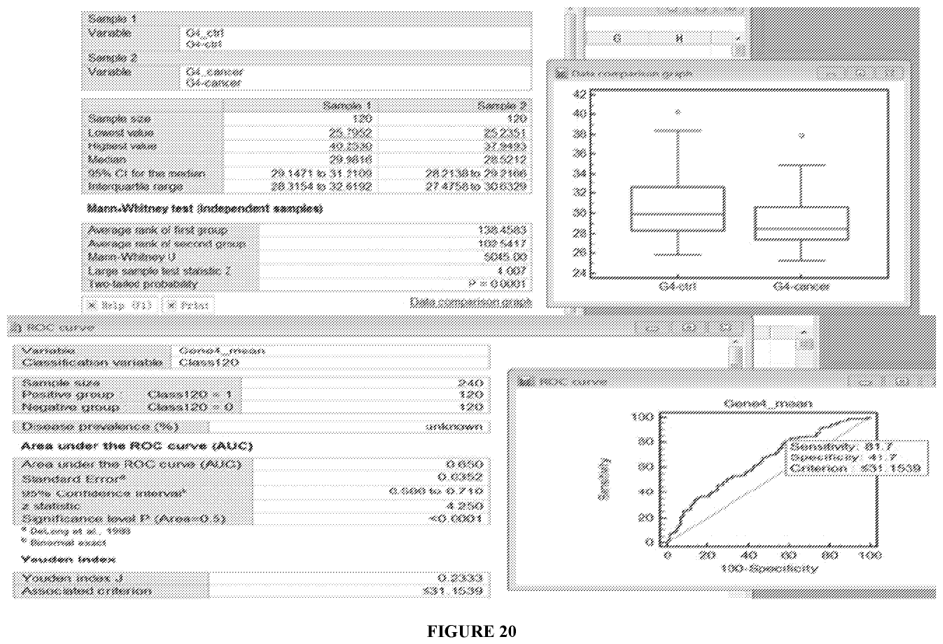

FIG. 18B shows the results of a RT-qPCR-based secondary validation study for Gene 4. Gene 4 was not one of the largest genetic contributors for breast cancer in saliva samples. The data showed good separation of cancer patients from control patients, and exhibited a sensitivity level of 81.7%, specificity level 41.7%, and a p-value that was less than 0.0001. FIG. 20 shows parameters and results of the biomarker validation study for Gene 4.

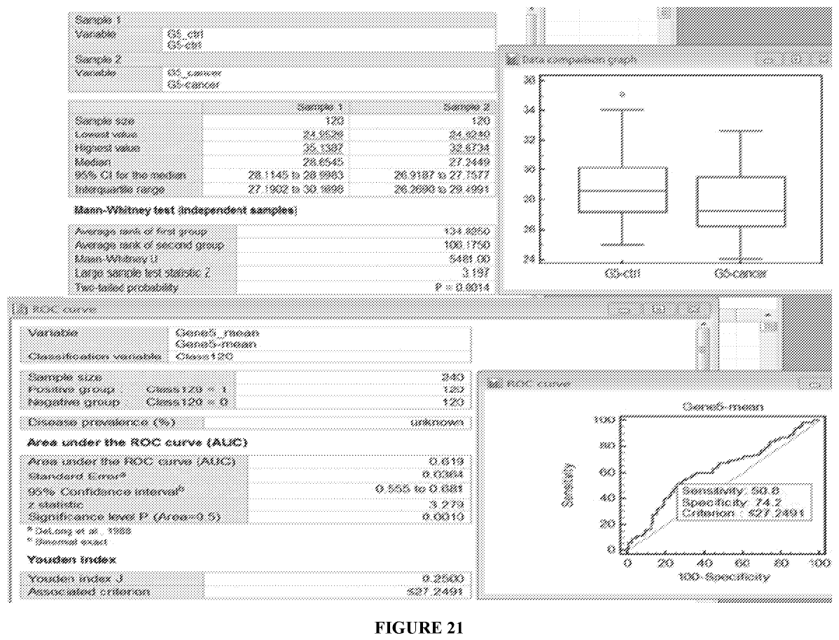

FIG. 18C shows the results of a RT-qPCR-based secondary validation study for Gene 5. Gene 5 was not one of the largest genetic contributors for breast cancer in saliva samples. The data showed good separation of cancer patients from control patients, and exhibited a sensitivity level of 50.8%, specificity level of 74.2%, and a p-value of 0.0014. FIG. 21 shows parameters and results of the biomarker validation study for Gene 5.

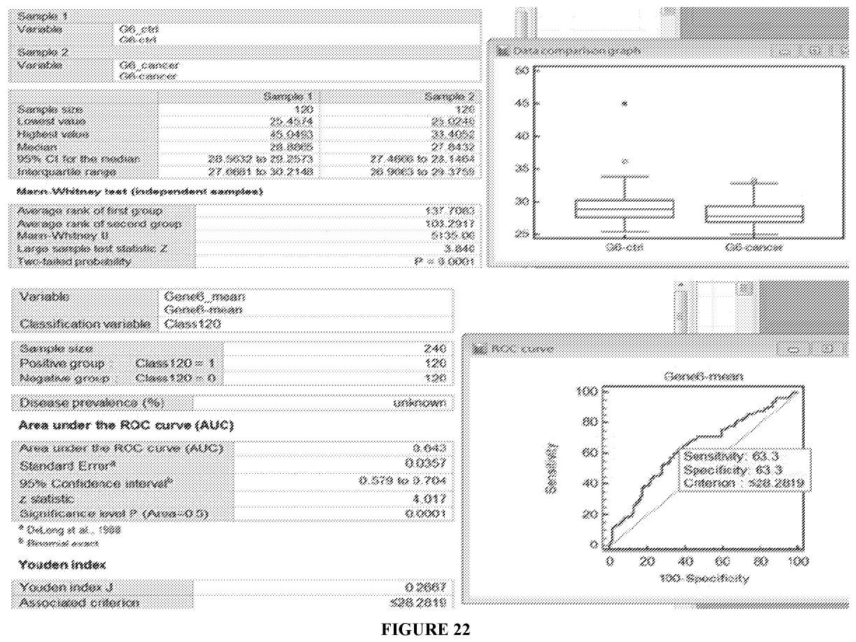

FIG. 18D shows the results of a RT-qPCR-based secondary validation study for Gene 6. Gene 6 was not one of the largest genetic contributors for breast cancer in saliva samples. The data showed good separation of cancer patients from control patients, and exhibited a sensitivity level of 63.3%, specificity of 63.3%, and a p-value of 0.0001. FIG. 22 shows parameters and results of the biomarker validation study for Gene 6.

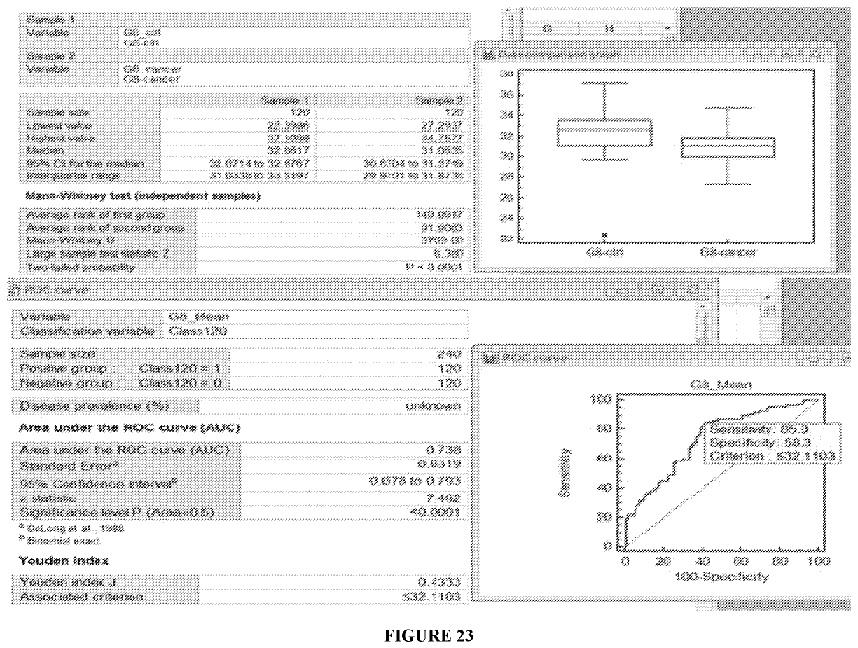

FIG. 18E shows the results of a RT-qPCR-based secondary validation study for Gene 8. Gene 8 was not one of the largest genetic contributors for breast cancer in saliva samples. The data showed good separation of cancer patients from control patients, and exhibited a sensitivity of about 85%, specificity of 58.5%, and a p-value that was less than 0.0001. FIG. 23 shows parameters and results of the biomarker validation study for Gene 8.

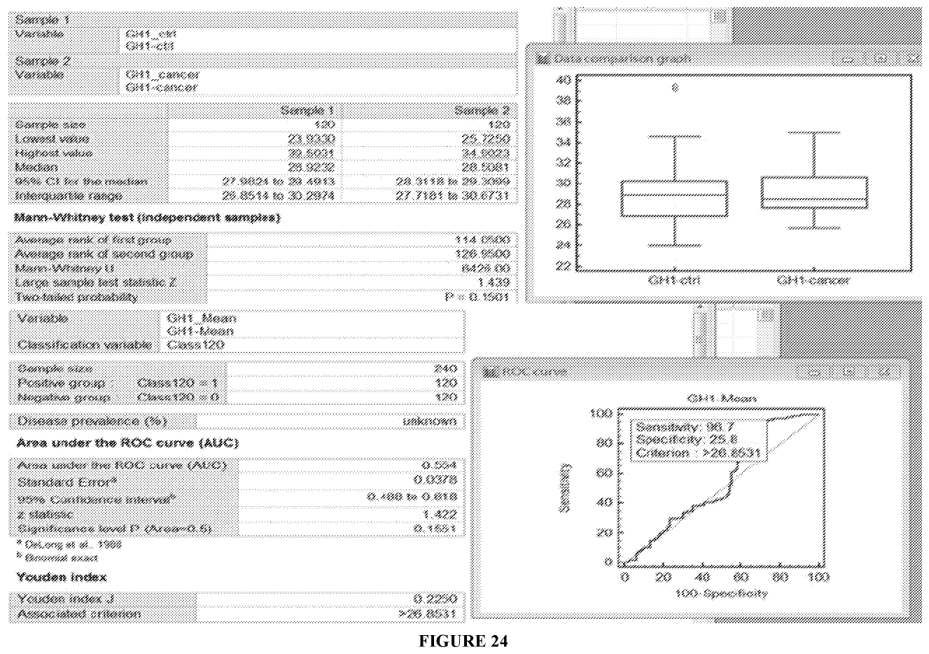

FIG. 24 shows the results of a RT-qPCR-based secondary validation study for the housekeeping gene G-H1. The data showed good separation of cancer patients from control patients, and exhibited sensitivity of 96.7%, specificity of 25.8%, and a p-value of 0.1551.

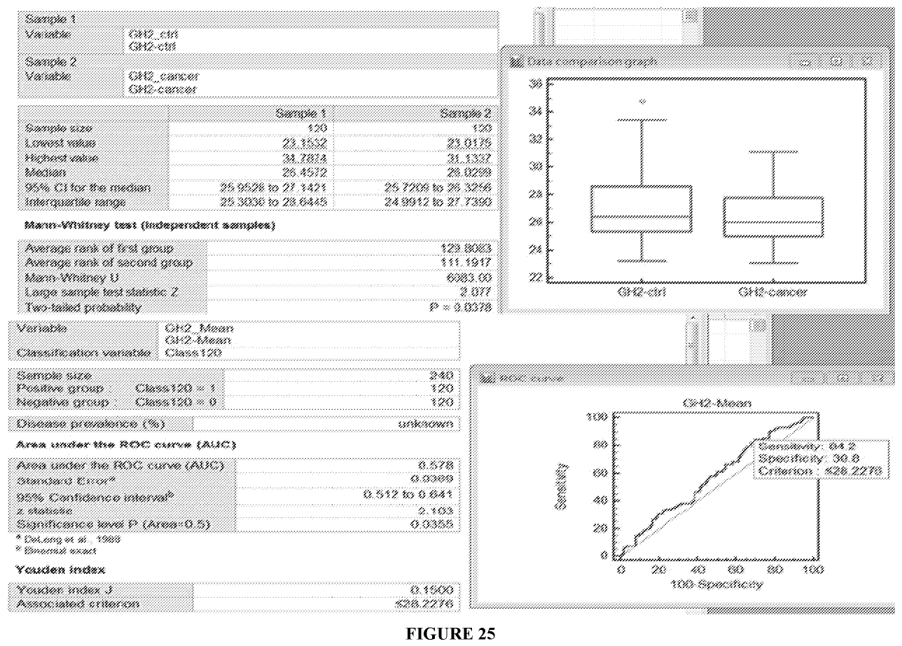

FIG. 25 shows the results of a RT-qPCR-based secondary validation study for the housekeeping gene G-H2. The data showed good separation of cancer patients from control patients, and exhibited sensitivity of 84.2%, specificity of 30.8%, and a p-value of 0.0355.

TABLE 2 shows the primers that were used to assay the 9 biomarker genes and 2 housekeeping genes. The data demonstrated that multiple genes showed individual significance in the large cohort study. Initial analysis showed significance when biomarkers were used in combination with additional biomarkers. Data showed that gene 2, gene 7, and gene 9 from the 9-gene biomarker panel contributed most to the test's specificity, for example, by correctly rejecting cancer or identifying negative samples correctly as normal. Gene 4 and gene 7 from the 9-gene biomarker panel contributed the most to the sensitivity, for example, by correctly rejecting normal samples or identifying positive samples correctly as cancer. The test's sensitivity and specificity were calculated using Medcalc Software. The sensitivity and specificity of the methods can be increased further by performing the tests as a companion to mammograms.

TABLE 2

| | | | | | | | | Self |
|---|---|---|---|---|---|---|---|---|
| | | | | | | | GC | comple- |
| | | 5′-3′ | Length | Start | Stop | Tim | % | mentarity |
| Gene 1 | Fl | CCTCCTAGAGGGTTGTAT | 22 | 230 | 251 | 59.9 | 54.55 | 6 |
| | | TGCC | | | | | | |
| AI424847 | R1 | AGGGAGGCTTGGAAGAG | 21 | 331 | 311 | 60.2 | 52.38 | 4 |
| | | AGAA | | | | | | |
| 243218_at | F2 | CAGTCGGAACCACATCCT | 20 | 177 | 196 | 60.11 | 60 | 3 |
| | | CC | | | | | | |
| | R2 | CTGTCTGACTGGTGGTCA | 21 | 385 | 365 | 59.65 | 52.38 | 5 |
| | | CAT | | | | | | |
| Gene 2 | Fl | CTTGTCATAGCCAAGCAC | 20 | 63 | 82 | 59.9 | 55 | 6 |
| | | GC | | | | | | |
| AW451259 | R1 | TGTCCCATGGAGGTAGGG | 20 | 288 | 269 | 60.03 | 60 | 6 |
| | | AG | | | | | | |
| 241371_at | F2 | GCTTCTTGGATTGACCTG | 20 | 84 | 103 | 59.19 | 55 | 3 |
| | | GC | | | | | | |
| | R2 | GCCTCGTGGTTCAATCCT | 20 | 253 | 234 | 60.74 | 60 | 4 |
| | | CC | | | | | | |
| Gene 3 | Fl | GGGGAACCTAACCGAGTC | 20 | 302 | 321 | 60.62 | 60 | 3 |
| | | CT | | | | | | |
| BF724944 | R1 | GATTACTTGGGGCACGCT | 20 | 480 | 461 | 60.68 | 55 | 2 |
| | | GT | | | | | | |
| 23874_at | F2 | TCCTGAGAGTGTAAGCCA | 20 | 318 | 337 | 59.1 | 55 | 3 |
| | | GC | | | | | | |
| | R2 | GACAGGATGATCCAGCCC | 20 | 569 | 550 | 59.16 | 55 | 7 |
| | | TT | | | | | | |
| Gene 4 | Fl | GTGGAGGTTCAGGACCAA | 20 | 1231 | 1250 | 59.96 | 60 | 5 |
| | | GG | | | | | | |
| AL162060 | R1 | CTCTCGGCATCAGCCTTC | 20 | 1520 | 1501 | 59.89 | 55 | 3 |
| | | AT | | | | | | |
| 212772 x at | F2 | CGCCCCTAATTGTGCCAA | 20 | 1307 | 1326 | 59.83 | 55 | 4 |
| | | AG | | | | | | |
| | R2 | TCTCGGCATCAGCCTTCA | 20 | 1519 | 1500 | 59.89 | 55 | 3 |
| | | TC | | | | | | |
| Gene 5 | Fl | GCATCTCCGGCCTCATCT | 20 | 137 | 156 | 60.04 | 60 | 4 |
| | | AC | | | | | | |
| NM_021968 | R1 | AGAAAGGGACGCTCAACC | 20 | 324 | 305 | 60.25 | 55 | 3 |
| | | AC | | | | | | |
| 208580 x at | F2 | CGCCGTGACCTATACAGA | 20 | 207 | 226 | 60.32 | 60 | 4 |
| | | GC | | | | | | |
| | R2 | CACCGAAACCGTAGAGGG | 20 | 307 | 288 | 60.39 | 60 | 4 |
| | | TG | | | | | | |
| Gene 6 | Fl | CCGACTGCTGTGAGAGTG | 20 | 320 | 339 | 59.68 | 55 | 4 |
| | | AA | | | | | | |
| NM_014357 | R1 | AGGTGCTCCATCAAGTGC | 20 | 545 | 526 | 59.89 | 50 | 4 |
| | | AA | | | | | | |
| 207710_at | F2 | ACAGCCTGATGCTTAACC | 21 | 434 | 454 | 59.64 | 47.62 | 5 |
| | | CTT | | | | | | |
| | R2 | GTGCTCCATCAAGTGCAA | 21 | 543 | 523 | 59.39 | 47.62 | 4 |
| | | AGT | | | | | | |
| Gene 7 | Fl | AAACGGAGACCCCGAAGT | 20 | 481 | 500 | 59.53 | 50 | 5 |
| | | TT | | | | | | |
| NM_006020 | R1 | CCACCCAGGAGAAAGATG | 20 | 782 | 763 | 60.39 | 60 | 3 |
| | | GC | | | | | | |
| 205621_at | F2 | ACCTTTCCCTTCTGACCT | 20 | 579 | 598 | 58.93 | 55 | 3 |
| | | GG | | | | | | |
| | R2 | AGCTGAATGACAGCAAGG | 20 | 751 | 732 | 59.6 | 50 | 4 |
| | | GT | | | | | | |
| Gene 8 | Fl | TGGAGGTCTTTGCCACCA | 20 | 1949 | 1968 | 59.52 | 50 | 4 |
| | | AT | | | | | | |
| AK092120 | R1 | AATTGCCTCTTCTGCTCC | 20 | 2094 | 2075 | 60.03 | 55 | 4 |
| | | CC | | | | | | |
| 1566840_at | F2 | CACCAATGGGAGATGAGC | 20 | 1962 | 1981 | 59.74 | 55 | 5 |
| | | CA | | | | | | |
| | R2 | GCTAAATGGCCCTCTCCT | 20 | 2072 | 2053 | 59.89 | 60 | 4 |
| | | CC | | | | | | |
| Gene 9 | Fl | TGTAAACAGCCAGACGTG | 20 | 374 | 393 | 60.25 | 55 | 4 |
| | | GG | | | | | | |
| AK022132 | R1 | CATAGCGCTCATGGCCAA | 20 | 479 | 460 | 59.97 | 55 | 8 |
| | | AC | | | | | | |
| 1565694_at | F2 | GTGTAAACAGCCAGACGT | 20 | 373 | 392 | 59.13 | 55 | 4 |
| | | GG | | | | | | |
| | R2 | CTGGAAAGCCCCGTTCTC | 20 | 496 | 477 | 60.04 | 55 | 3 |
| | | AT | | | | | | |
| Housekeeping | | | | | | | | |
| genes | | | | | | | | |
| Gene 1-1 | Fl | ACCGGCACCATCAAGCT | 17 | | | 54.9 | 59 | |
| TFRC | R1 | TGATCACGCCAGACTTTG | 19 | | | 57.5 | 53 | |
| | | C | | | | | | |
| | F2 | CTCGTGAGGCTGGATCTC | 21 | 744 | 764 | 59.79 | 52.38 | 4 |
| | | AAA | | | | | | |
| | R2 | TCACGCCAGACTTTGCTG | 20 | 834 | 815 | 60.6 | 55 | 3 |
| | | AG | | | | | | |
| Gene 1-2 | Fl | CCATCATGAAGTGTGACG | 21 | 926 | 946 | 61.2 | 52 | |
| | | TGG | | | | | | |
| ATCB | R1 | GTCCGCCTAGAAGCATTT | 21 | 1198 | 1218 | 63.2 | 57 | |
| | | GCG | | | | | | |
| | F2 | TTGCCGACAGGATGCAGA | 23 | 1010 | 1031 | 64.2 | 55 | |
| | | AGGA | | | | | | |
| | R2 | AGGTGGACAGCGAGGCC | 23 | 1117 | 1138 | 67.9 | 64 | |
| | | AGGAT | | | | | | |
| Gene 1-3 | Fl | AGGCTGGGGCATACAAAA | | | | | | |
| | | CCA | | | | | | |
| AB032983 | R1 | CGCCATCCTCTGTCCTTC | 20 | 1877 | 1858 | 60.18 | 60 | 3 |
| | | AG | | | | | | |
| 212686_at | F2 | GCCCGGGTAATGGCAACT | 20 | 1552 | 1571 | 60.18 | 55 | 6 |
| | | AT | | | | | | |
| | R2 | CGTCCCAGAGTCCATCAG | 20 | 1738 | 1719 | 59.83 | 60 | 3 |
| | | TG | | | | | | |

### Example 4: Correlating Transcriptional Level of Genes in the Biomarker Panel Assay with Known Involvement to Oncology

Biomarker levels (e.g., transcriptional levels of the 9 biomarker genes from EXAMPLE 2) are correlated with those genes known to be involved in breast cancer formation and progression. Fold change differences related to the biomarkers are examined between cancer and healthy subjects and subclasses related to age, race, physical condition, breast cancer type or stage, and breast tissue density. This information is used to guide a gene ontology search of genes and related pathways known to be involved in breast cancer formation and progression. Based on this information, rankings and weightings of the expression levels of the biomarkers are determined to improve sensitivity of the test.

Patient information and saliva samples for 30 patients (15 cancer patients and 15 control subjects) are obtained. The saliva samples are shipped in Oragene RE-100 cups, which contain RNase inhibitors to stabilize the RNA in saliva for 60 days at room temperature. RNA is extracted from the saliva. qPCR is run in duplicate on each sample. Differences in gene expression levels for the gene panel (e.g., the 9 biomarker genes identified in EXAMPLE 2) are examined between healthy and cancer subjects based on fold change differences and p-values (t-test). The data obtained from the gene panel are used as the baseline.

Changes in gene expression in subclasses related to patient information are then analyzed. Changes in gene expression of the subclasses are used to guide the gene ontology search. For example, if age creates the largest delta from the baseline, the relationships of the 9 biomarker genes to genes involved in age-related pathways that also have a relation to breast cancer are analyzed. Gene ontology tools (e.g., AmiGO 2 and Gene at NCBI) are used for the gene ontology search. The gene ontology search procedure is carried out for the three largest deltas from the healthy subject-cancer subject baseline. Based on the results from the three guided gene ontology searches, three ranking and weighting regimes are calculated. The three weighting regimes are then tested against unweighted scoring of the 30 samples for accuracy, sensitivity, and specificity, and the results are compared. The weighting regime raises the sensitivity to greater than 90%, improves overall accuracy of the assay, and keeps the specificity level at or above 97%.

### Example 5: Examination of the mRNA Content of Breast Cancer-Associated Exosomes

The mRNA contents of exosomes released from immortalized breast cancer cell lines (e.g., MDA-MB-231 and MCF7) grown in culture with and without standard-of-care chemotherapeutic are examined. Data obtained from the mRNA contents of exosomes are used to further refine the weighting of gene expression values and to improve test result measures.

MDA-MB-231 and MCF7 immortalized breast cancer cell lines can release mRNA-containing exosome-like vesicles into the growth media. The transcriptional level of genes in the biomarker panel (e.g., the 9 biomarker genes identified in EXAMPLE 2) is examined in exosomes released from immortalized breast cancer cell lines (e.g., MDA-MB-231 and MCF7) cultured with and without doxorubicin (i.e., a standard-of-care chemotherapeutic). The samples are analyzed using standard laboratory techniques, such as qPCR. The differences in expression levels are analyzed, for example, as discussed in EXAMPLE 4. Based on the analysis, a refined ranking and weighting regime is derived. The refined weighting regime is tested against both the weighted scoring regime from EXAMPLE 4 and an unweighted scoring regime for accuracy, sensitivity, and specificity using the data from the 30 samples in EXAMPLE 4. The results are then compared.

Differences are observed in the expression levels of genes in the biomarker panel (e.g., for the 9 gene-assay from EXAMPLE 2) in cells cultured with and without doxorubicin. The differences in expression levels of genes can provide further evidence of an exosomal mechanism involved in breast cancer detection in saliva. The refined weighting regime can raise the sensitivity above 90%, and improve the overall accuracy of the biomarker assay without significantly affecting assay specificity.

### Example 6: Testing Predictive Power of the Biomarker Panel Using Blinded Patient Samples (n=30)

Blinded saliva samples (e.g., 30 samples with unknown cancer, control information) are analyzed and scored as in EXAMPLE 4 using any new weighting optimizations gleaned from EXAMPLE 4 and EXAMPLE 5.

### Example 7: Workflow for Saliva-Based Diagnostic Assay

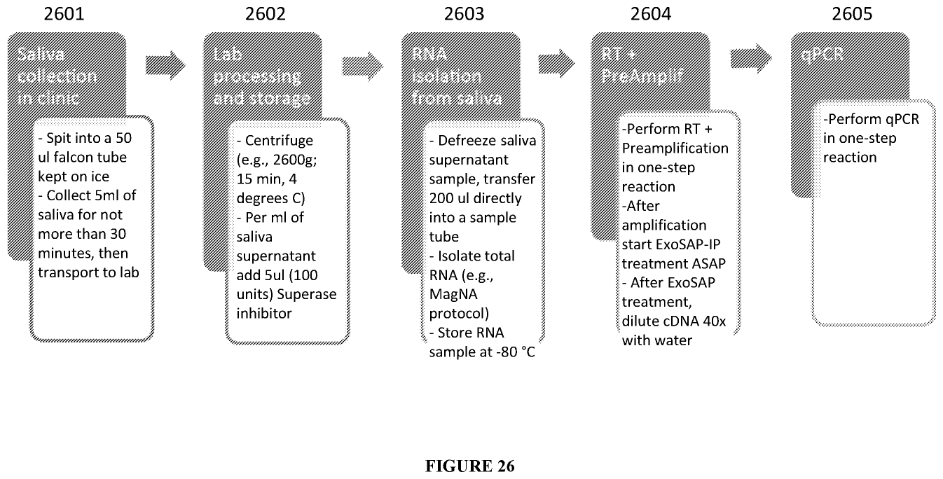

FIG. 26 illustrates an optimized work flow for the saliva gene test. 5 mL of saliva was collected in a 50 mL collection tube within 30 minutes, and the tube was transported to a diagnostic lab (2601). The sample was centrifuged at 2600 g for 15 min at 4° C. The supernatant was collected. 5 μL (i.e., 100 units) of superase inhibitor was added per mL of the saliva supernatant, and the sample was stored (2602). RNA was then isolated from the saliva sample (2603). The saliva supernatant sample was thawed. 200 μL of the thawed sample was transferred directly into a sample tube. Total RNA was isolated according to a standard MagNA protocol. RNA samples were stored at −80° C. The RNA was reverse transcribed and pre-amplified in a one-step reaction (2604) using experimental parameters shown TABLE 3 and TABLE 4.

TABLE 3

| | Per tube | Number of tubes | Total |
|---|---|---|---|
| StarScript II RT Mix | 1 | 96 | 96 |
| Primers 3 housekeeping + | 1.44 | 96 | 138.24 |
| 9 pairs (100 μM/each) | | | |
| 2x Reaction mix | 10 | 96 | 960 |
| mRNA | 4 | 96 | |
| H₂O | 3.56 | 96 | 341.76 |
| TOTAL Volume | 20 | | |

TABLE 4

| Time | Temp. | Cycle | |
|---|---|---|---|
| 1 min | 60 | 1 | RT |
| 30 min | 50 | | |
| 2 min | 95 | | |
| 15 sec | 95 | 15 | Preamp |
| 30 sec | 50 | | |
| 10 sec | 60 | | |
| 10 sec | 72 | | |
| 10 min | 72 | 1 | Extension |
| Hold | 4 | 1 | |

After amplification, the amplified products were purified using ExoSAP-IT treatment, for example, to eliminate unconsumed dNTPs and primers remaining in the PCR product mixture that could interfere with downstream applications (e.g., qPCR and sequencing). After purification, the cDNA was diluted about 40 fold. qPCR was performed in a one-step reaction (2605) using experimental conditions shown TABLE 5 and TABLE 6.

TABLE 5

| | per tube | Number of tubes | Total |
|---|---|---|---|
| Inner primer mix | 0.18 | 96 | 17.28 |
| (100 μM/each) | | | |
| 2x qPCR Mix | 5 | 96 | 480 |
| Water | 0.82 | 96 | 78.72 |
| cDNA | 4 | | |
| TOTAL Volume | 10 | | |

TABLE 6

| Time | Temp. | Cycle | |
|---|---|---|---|
| 10 min | 95 | 1 | Hot start |
| 15 sec | 95 | 40 | qPCR |
| 30 sec | 60 | | |
| 15 sec | 95 | 1 | Dissociation |
| 1 min | 60 | | |
| 15 sec | 95 | | |
| 15 sec | 60 | | |

### Example 8: Evaluate Gene Expression Profiles in Saliva for Breast Cancer Associated Genes

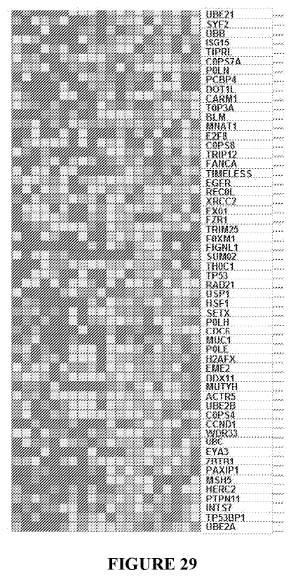

Using RNA collected from saliva from 10 patients with breast cancer and 10 normal patients, a correlation to the cancer and normal phenotype was computed. This was used to determine genes that are differentially expressed between cancer and normal samples. FIG. 29 illustrates the results of the study in the form of a heatmap. In FIG. 29, the first 10 columns show data from saliva of cancer patients and the second ten columns show data from saliva of normal samples. The boxes in each row show the expression of the gene in the 20 patients. Blue box indicated gene expression was down. Red box indicated gene expression was up.

Genes that were determined to be differentially expressed were analyzed to determine their enrichment in the hallmarks of cancer (e.g., as annotated by the GO ontology) using test Kolmgorov-Smirnoff statistic with a P-value cutoff of 0.05. FIG. 28 illustrates genes that were found to be differentially expressed and corresponded to hallmarks of cancer. These included TNFRSF10A, ABCA1/2, DTYMK, and ALKBH1, which were independently identified as candidate biomarkers in EXAMPLE 2. Thus, this study validated that candidate genes identified in EXAMPLE 2 and shown in FIG. 4 can be used as biomarkers for breast cancer detection from saliva.

### Example 9: Diagnostic Test for Breast Cancer

A female subject undergoes a mammogram. The subject is notified that she has dense breast tissue. The mammogram shows a negative indication for cancer. Because the subject has dense breast tissue, the healthcare provider recommends a breast cancer biomarker assay (e.g., the 9-gene assay described in EXAMPLE 4).

Following the healthcare provider's recommendation, the subject spits into a cup. The saliva sample is analyzed using methods of the disclosure to determine the transcriptional level of genes from the biomarker panel. The subject is given a diagnosis based on analysis of data from the biomarker assay.

### Example 10: Companion Diagnostic

FIG. 2 illustrates the use of a saliva-based biomarker assay in conjunction with mammogram imaging for accurate cancer diagnosing. A female subject (201) undergoes a mammogram (202) and submits a saliva sample (204) to the healthcare provider. The mammogram is analyzed (203) to detect cancer. Simultaneously, the saliva sample is analyzed (205) using methods of the disclosure to determine the transcriptional level of genes from a biomarker panel (e.g., the 9-gene assay described in EXAMPLE 4). The subject is given a diagnosis (206) based on analysis of the mammogram and data from the biomarker assay.

### Example 11: Breast Cancer Diagnostic

A subject would like to be annually screened for breast cancer. The subject provides a saliva sample by mail or in person, in advance of imaging. The subject's sample is analyzed for biomarker panel. Based on the results (e.g., communicated by mail or in person), the biomarker classifies the subject into risk categories. These categories can be used to specify the risk of cancer and the frequency of follow-up. The results can also provide recommendation on additional screening by mammogram, MRI, and/or more extensive surveillance if saliva results indicate very high risk.

### Example 12: Breast Cancer Diagnostic

A subject comes to a healthcare provider for an annual screening mammogram and provides a saliva sample at the same time. The two tests are analyzed separately. The results are combined to generate a single combined probability score for cancer, which can be a more robust estimate of breast cancer risk than either test alone.

### Example 13: Breast Cancer Diagnostic

A subject comes to a healthcare provider for annual screening and obtains a mammogram with an “ambiguous result” reading. Approximately 1/7 mammograms can be ambiguous. The subject provides a saliva sample, which is analyzed using a biomarker assay of the disclosure. Results from the biomarker assay and mammogram are combined to prioritize the subject for ongoing follow-up such as repeat mammogram, MRI, biopsy, or increased frequency of surveillance by saliva sample testing, or mammogram, or both.

### Example 14: Cancer Screening

A subject comes for screening and provides a sample. The sample is analyzed and the test results identify a cancer in the patient's body. The subject undergoes follow-on testing such as a biomarker assay of the disclosure to locate the cancer to a specific body tissue such as breast.

### Example 15: Avoidance of Mammogram

A subject wishes to avoid mammogram. Mammograms can have high false negative and false positive rates, such as for subjects with dense breasts, or who are young (e.g., age range of 18 to 40, or below 40, or below 35), or at high-risk of breast cancer. Younger subjects can have higher frequency of dense breasts. The subject is 34 and has dense breast tissue. The subject undergoes a saliva-based biomarker assay of the disclosure. The subject is given a risk score for breast cancer, recommendation for additional testing, and frequency of future screening.

### EMBODIMENTS

The following non-limiting embodiments provide illustrative examples of the invention, but do not limit the scope of the invention.

### Embodiment 1

A method comprising:
a) performing a screening test on a subject, wherein the screening test comprises evaluating the subject for a risk of developing a health condition;
b) obtaining a biological sample of the subject;
c) quantifying a sample level of a biomarker in the biological sample of the subject;
d) comparing the sample level of the biomarker to a reference level of the biomarker;
e) combining the result of the screening test and the comparing; and
f) determining a health state of the subject based on the combining.

### Embodiment 2

The method of embodiment 1, wherein the screening test comprises imaging a breast tissue of the subject.

### Embodiment 3

The method of any one of embodiments 1-2, wherein the imaging is performed using a mammogram.

### Embodiment 4

The method of any one of embodiments 1-3, wherein the screening test comprises quantifying a sample level of a cell-free nucleic acid in the subject.

### Embodiment 5

The method of any one of embodiments 1-4, wherein the cell-free nucleic acid is cell-free RNA.

### Embodiment 6

The method of any one of embodiments 1-4, wherein the cell-free nucleic acid is cell-free DNA.

### Embodiment 7

The method of any one of embodiments 1-6, wherein the cell-free nucleic acid is specific to a tissue of the subject.

### Embodiment 8

The method of any one of embodiments 1-7, wherein the tissue is a breast tissue.

### Embodiment 9

The method of any one of embodiments 1-7, wherein the cell-free nucleic acid is from a biofluid.

### Embodiment 10

The method of any one of embodiments 1-7 and 9, wherein the biofluid is selected from the group consisting of: blood, a blood fraction, serum, plasma, saliva, sputum, urine, semen, a transvaginal fluid, a cerebrospinal fluid, sweat, bile, cyst fluid, tear, breast aspirate, and breast fluid.

### Embodiment 11

The method of any one of embodiments 1-10, wherein the screening test comprises a genetic test.

### Embodiment 12

The method of any one of embodiments 1-11, wherein the genetic test comprises testing for a mutation in a breast cancer susceptibility gene.

### Embodiment 13

The method of any one of embodiments 1-12, wherein the genetic test comprises testing for a mutation in a gene selected from the group consisting of: ATM, BARD1, BRCA1, BRCA2, BRIP1, CASP8, CDH1, CHEK2, CTLA4, CYP19A1, FGFR2, H19, LSP1, MAP3K1, MRE11, NBN, PALB2, PTEN, RAD51, RAD51C, STK11, TERT, TOX3, TP53, XRCC2, XRCC3, and any combination thereof.

### Embodiment 14

The method of any one of embodiments 1-13, wherein the health condition is cancer.

### Embodiment 15

The method of any one of embodiments 1-14, wherein the cancer is breast cancer.

### Embodiment 16

The method of any one of embodiments 1-15, wherein the biological sample is a biofluid.

### Embodiment 17

The method of any one of embodiments 1-16, wherein the biofluid is saliva.

### Embodiment 18

The method of any one of embodiments 1-16, wherein the biofluid is blood.

### Embodiment 19

The method of any one of embodiments 1-18, wherein the biofluid is selected from the group consisting of: blood, a blood fraction, serum, plasma, saliva, sputum, urine, semen, a transvaginal fluid, a cerebrospinal fluid, sweat, bile, cyst fluid, tear, breast aspirate, and breast fluid.

### Embodiment 20

The method of any one of embodiments 1-19, wherein the biomarker is selected from the group consisting of: a nucleic acid, peptide, protein, lipid, antigen, carbohydrate and proteoglycan.

### Embodiment 21

The method of any one of embodiments 1-20, wherein the biomarker is a nucleic acid, wherein the nucleic acid is DNA or RNA.

### Embodiment 22

The method of any one of embodiments 1-21, wherein the nucleic acid is RNA, wherein the RNA is selected from the group consisting of: mRNA, miRNA, snoRNA, snRNA, rRNAs, tRNAs, siRNA, hnRNA, and shRNA.

### Embodiment 23

The method of any one of embodiments 1-22, wherein the RNA is mRNA.

### Embodiment 24

The method of any one of embodiments 1-22, wherein the RNA is miRNA.

### Embodiment 25

The method of any one of embodiments 1-21, wherein the biomarker is a nucleic acid, wherein the nucleic acid is DNA, wherein the DNA is selected from the group consisting of: double-stranded DNA, single-stranded DNA, complementary DNA, and noncoding DNA.

### Embodiment 26

The method of any one of embodiments 1-21, wherein the biomarker is a cell-free nucleic acid.

### Embodiment 27

The method of any one of embodiments 1-24, wherein the cell-free nucleic acid is a cell-free RNA.

### Embodiment 28

The method of any one of embodiments 1-21, wherein the cell-free RNA is cell free mRNA or cell free miRNA.

### Embodiment 29

The method of any one of embodiments 1-20, wherein the biomarker is a cell-free DNA.

### Embodiment 30

The method of any one of embodiments 1-20, wherein the biomarker is of exosomal origin.

### Embodiment 31

The method of any one of embodiments 1-20, wherein the biomarker is a protein.

### Embodiment 32

The method of any one of embodiments 1-20, wherein the biomarker is a gene in a breast cancer pathway.

### Embodiment 33

The method of any one of embodiments 1-20 and 30-32, wherein the biomarker is selected from the group consisting of: MCART1, LCE2B, HIST1H4K, ABCA2, TNFRSF10A, AK092120, DTYMK, Hs.161434, ALKBH1, and any combination thereof.

### Embodiment 34

The method of any one of embodiments 1-20 and 30-33, wherein the biomarker is HIST1H4K.

### Embodiment 35

The method of any one of embodiments 1-20 and 30-33, wherein the biomarker is TNFRSF10A.

### Embodiment 36

The method of any one of embodiments 1-20 and 30-35, wherein quantifying the sample level of the biomarker comprises quantifying at least two biomarkers, wherein the at least two biomarkers are selected from the group consisting of LCE2B, HIST1H4K, ABCA2, TNFRSF10A, AK092120, DTYMK, ALKBH1, MCART1, and Hs.161434.

### Embodiment 37

The method of any one of embodiments 1-20 and 30-36, wherein quantifying the sample level of the biomarker comprises quantifying two biomarkers, wherein the two biomarkers are HIST1H4K and TNFRSF10A.

### Embodiment 38

The method of any one of embodiments 1-37, wherein the determining the health state comprises determining the health state of the tissue of the subject.

### Embodiment 39

The method of any one of embodiments 1-38, wherein the tissue is breast tissue.

### Embodiment 40

The method of any one of embodiments 1-39, further comprising experimentally lysing an exosomal fraction of the biological sample to release the biomarker from the exosomal fraction.

### Embodiment 41

The method of any one of embodiments 1-40, wherein the reference level is obtained from a subject having breast cancer.

### Embodiment 42

The method of any one of embodiments 1-41, wherein the quantifying further comprises experimentally reverse transcribing the RNA.

### Embodiment 43

The method of any one of embodiments 1-42, wherein the quantifying further comprises performing a polymerase chain reaction.

### Embodiment 44

The method of any one of embodiments 1-43, wherein the wherein the PCR is quantitative PCR.

### Embodiment 45

The method of any one of embodiments 1-44, wherein the quantifying further comprises performing sequencing, wherein the sequencing comprises massively parallel sequencing.

### Embodiment 46

The method of any one of embodiments 1-45, wherein the quantifying the sample level of biomarker is performed with an accuracy of at least 90%.

### Embodiment 47

The method of any one of embodiments 1-46, wherein the quantifying the sample level of biomarker is performed with an accuracy of at least about: 90%, 91%, 92%, 93%, 94%, 95%, 95%, 97%, 98%, or 99%.

### Embodiment 48

The method of any one of embodiments 1-47, wherein the quantifying the sample level of biomarker is performed with a sensitivity of at least about 80%.

### Embodiment 49

The method of any one of embodiments 1-48, wherein the quantifying the sample level of biomarker is performed with a sensitivity of at least: 80%, 85%, 90%, 91%, 92%, 93%, 94%, 95%, 95%, 97%, 98%, or 99%.

### Embodiment 50

The method of any one of embodiments 1-49, wherein the quantifying the sample level of biomarker is performed with a specificity of at least 90%.

### Embodiment 51

The method of any one of embodiments 1-50, wherein the quantifying the sample level of biomarker is performed with a specificity of at least: 90%, 91%, 92%, 93%, 94%, 95%, 95%, 97%, 98%, or 99%.

### Embodiment 52

A method comprising:
a) obtaining a saliva sample of a subject;
b) experimentally lysing an exosome fraction of the saliva sample to release a biomarker;
c) quantifying a sample level of the biomarker; and
d) comparing the sample level of the biomarker to a reference level of the biomarker, wherein the reference level is obtained from a subject having breast cancer.

### Embodiment 53

The method of embodiment 52, further comprising an additional test, wherein the additional test comprises evaluating the subject for a risk of developing breast cancer.

### Embodiment 54

The method of any one of embodiments 52-53, further comprising combining the result of the additional test and the comparing in step d.

### Embodiment 55

The method of any one of embodiments 52-54, further comprising determining a breast cancer state of the subject based on the combining.

### Embodiment 56

The method of any one of embodiments 52-55, wherein the additional test comprises imaging a breast tissue of the subject.

### Embodiment 57

The method of any one of embodiments 52-56, wherein the imaging is performed using a mammogram.

### Embodiment 58

The method of any one of embodiments 52-57, wherein the additional test comprises quantifying a sample level of a cell-free nucleic acid in the subject.

### Embodiment 59

The method of any one of embodiments 52-58, wherein the cell-free nucleic acid is cell-free RNA or cell free DNA.

### Embodiment 60

The method of any one of embodiments 52-59, wherein the cell-free nucleic acid is specific to a tissue of the subject.

### Embodiment 61

The method of any one of embodiments 52-60, wherein the tissue is a breast tissue.

### Embodiment 62

The method of any one of embodiments 52-60, wherein the cell-free nucleic acid is from a biofluid.

### Embodiment 63

The method of any one of embodiments 52-60 and 62, wherein the biofluid is selected from the group consisting of: blood, a blood fraction, serum, plasma, saliva, sputum, urine, semen, a transvaginal fluid, a cerebrospinal fluid, sweat, bile, cyst fluid, tear, breast aspirate, and breast fluid.

### Embodiment 64

The method of any one of embodiments 52-63, wherein the biomarker is selected from the group consisting of: a nucleic acid, peptide, protein, lipid, antigen, carbohydrate, and proteoglycan.

### Embodiment 65

The method of any one of embodiments 52-64, wherein the biomarker is a nucleic acid.

### Embodiment 66

The method of any one of embodiments 52-65, wherein the nucleic acid is RNA.

### Embodiment 67

The method of any one of embodiments 52-66, wherein the RNA is mRNA.

### Embodiment 68

The method of any one of embodiments 52-66, wherein the RNA is miRNA.

### Embodiment 69

The method of any one of embodiments 52-65, wherein the nucleic acid is DNA.

### Embodiment 70

The method of any one of embodiments 52-69, wherein the biomarker is a gene in a breast cancer pathway.

### Embodiment 71

The method of any one of embodiments 52-70, wherein the biomarker is selected from the group consisting of: LCE2B, HIST1H4K, ABCA2, TNFRSF10A, AK092120, DTYMK, Hs.161434, ALKBH1, MCART1, and any combination thereof.

### Embodiment 72

The method of any one of embodiments 52-71, wherein the biomarker is HIST1H4K.

### Embodiment 73

The method of any one of embodiments 52-71, wherein the biomarker is TNFRSF10A.

### Embodiment 74

The method of any one of embodiments 52-73, wherein the quantifying the sample level of the biomarker comprises quantifying at least two biomarkers, wherein the at least two biomarkers are selected from the group consisting of: LCE2B, HIST1H4K, ABCA2, TNFRSF10A, AK092120, DTYMK, ALKBH1, MCART1, and Hs.161434.

### Embodiment 75

The method of any one of embodiments 52-74, wherein the quantifying the sample level of the biomarker comprises quantifying two biomarkers, wherein the two biomarkers are HIST1H4K and TNFRSF10A.

### Embodiment 76

The method of any one of embodiments 52-75, further comprising experimentally enriching the exosome fraction of the saliva sample prior to step b.

### Embodiment 77

The method of any one of embodiments 52-76, further comprising stabilizing the exosome fraction following experimentally enriching.

### Embodiment 78

The method of any one of embodiments 52-68 and 70-77, wherein the biomarker is a RNA, wherein the quantifying further comprises experimentally reverse transcribing the RNA.

### Embodiment 79

The method of any one of embodiments 52-78, wherein the quantifying further comprises performing a polymerase chain reaction.

### Embodiment 80

The method of any one of embodiments 52-79, wherein the wherein the PCR is quantitative PCR.

### Embodiment 81

The method of any one of embodiments 52-80, wherein the quantifying further comprises performing sequencing, wherein the sequencing comprises massively parallel sequencing.

### Embodiment 82

The method of any one of embodiments 52-81, wherein the quantifying the sample level of biomarker is performed with an accuracy of at least 90%.

### Embodiment 83

The method of any one of embodiments 52-82, wherein the quantifying the sample level of biomarker is performed with an accuracy of at least about: 90%, 91%, 92%, 93%, 94%, 95%, 95%, 97%, 98%, or 99%.

### Embodiment 84

The method of any one of embodiments 52-83, wherein the quantifying the sample level of biomarker is performed with a sensitivity of at least about 80%.

### Embodiment 85

The method of any one of embodiments 52-84, wherein the quantifying the sample level of biomarker is performed with a sensitivity of at least: 80%, 85%, 90%, 91%, 92%, 93%, 94%, 95%, 95%, 97%, 98%, or 99%.

### Embodiment 86

The method of any one of embodiments 52-85, wherein the quantifying the sample level of biomarker is performed with a specificity of at least 90%.

### Embodiment 87

The method of any one of embodiments 52-86, wherein the quantifying the sample level of biomarker is performed with a specificity of at least: 90%, 91%, 92%, 93%, 94%, 95%, 95%, 97%, 98%, or 99%.

### Embodiment 88

The method of any one of embodiments 52-87, further comprising a genetic test.

### Embodiment 89

The method of any one of embodiments 52-88, wherein the genetic test comprises testing for a mutation in a breast cancer susceptibility gene.

### Embodiment 90

The method of any one of embodiments 52-89, wherein the genetic test comprises testing for a mutation in a gene selected from the group consisting of: ATM, BARD1, BRCA1, BRCA2, BRIP1, CASP8, CDH1, CHEK2, CTLA4, CYP19A1, FGFR2, H19, LSP1, MAP3K1, MRE11, NBN, PALB2, PTEN, RAD51, RAD51C, STK11, TERT, TOX3, TP53, XRCC2, XRCC3, and any combination thereof.

### Embodiment 91

A method comprising:
a) performing a mammogram on a subject;
b) obtaining a saliva sample of the subject;
c) quantifying a sample level of a biomarker in the saliva sample of the subject, wherein the biomarker is of exosomal origin;
d) comparing the sample level of the biomarker to a reference level of the biomarker, wherein the reference level is obtained from a subject having breast cancer; and
e) combining the result of the mammogram and the comparing to determine a health state of the subject.

### Embodiment 92

The method of embodiment 91, wherein the mammogram result is negative for breast cancer in the subject.

### Embodiment 93

The method of any one of embodiments 91-92, further comprising identifying the negative result from the mammogram as a false negative based on the combining in step e.

### Embodiment 94

The method of embodiment 91, wherein the mammogram result is positive for breast cancer in the subject.

### Embodiment 95

The method of any one of embodiments 91 and 94, further comprising identifying a positive result from the mammogram as a false positive result based on the combining in step e.

### Embodiment 96

The method of embodiment 91, wherein the mammogram result is ambiguous for breast cancer in the subject.

### Embodiment 97

The method of any one of embodiments 91-96, wherein the biomarker is selected from the group consisting of: a nucleic acid, peptide, protein, lipid, antigen, carbohydrate, and proteoglycan.

### Embodiment 98

The method of any one of embodiments 91-97, wherein the biomarker is a nucleic acid.

### Embodiment 99

The method of any one of embodiments 91-98, wherein the nucleic acid is RNA.

### Embodiment 100

The method of any one of embodiments 91-99, wherein the RNA is mRNA.

### Embodiment 101

The method of any one of embodiments 91-99, wherein the RNA is miRNA.

### Embodiment 102

The method of any one of embodiments 91-98, wherein the nucleic acid is DNA.

### Embodiment 103

The method of any one of embodiments 91-102, wherein the biomarker is a gene in a breast cancer pathway.

### Embodiment 104

The method of any one of embodiments 91-103, wherein the biomarker is selected from the group consisting of: LCE2B, HIST1H4K, ABCA2, TNFRSF10A, AK092120, DTYMK, Hs.161434, ALKBH1, MCART1, and any combination thereof.

### Embodiment 105

The method of any one of embodiments 91-104, wherein the biomarker is HIST1H4K.

### Embodiment 106

The method of any one of embodiments 91-104, wherein the biomarker is TNFRSF10A.

### Embodiment 107

The method of any one of embodiments 91-106, wherein the quantifying the sample level of the biomarker comprises quantifying at least two biomarkers, wherein the at least two biomarkers are selected from the group consisting of LCE2B, HIST1H4K, ABCA2, TNFRSF10A, AK092120, DTYMK, ALKBH1, MCART1, and Hs.161434.

### Embodiment 108

The method of any one of embodiments 91-107, wherein the quantifying the sample level of the biomarker comprises quantifying two biomarkers, wherein the two biomarkers are HIST1H4K and TNFRSF10A.

### Embodiment 109

The method of any one of embodiments 91-108, further comprising lysing an exosome fraction of the saliva sample.

### Embodiment 110

The method of any one of embodiments 91-109, further comprising experimentally enriching the exosome fraction of the saliva sample prior to lysing.

### Embodiment 111

The method of any one of embodiments 91-110, further comprising stabilizing the exosome fraction following experimentally enriching.

### Embodiment 112

The method of any one of embodiments 91-101 and 103-111, wherein the biomarker is a RNA, wherein the quantifying further comprises experimentally reverse transcribing the RNA.

### Embodiment 113

The method of any one of embodiments 91-101 and 103-112, wherein the quantifying further comprises performing a polymerase chain reaction.

### Embodiment 114

The method of any one of embodiments 91-101 and 103-113, wherein the PCR is quantitative PCR.

### Embodiment 115

The method of any one of embodiments 91-114, wherein the quantifying further comprises performing sequencing, wherein the sequencing comprises massively parallel sequencing.

### Embodiment 116

The method of any one of embodiments 91-115, wherein the quantifying the sample level of biomarker is performed with an accuracy of at least 90%.

### Embodiment 117

The method of any one of embodiments 91-116, wherein the quantifying the sample level of biomarker is performed with an accuracy of at least about: 90%, 91%, 92%, 93%, 94%, 95%, 95%, 97%, 98%, or 99%.

### Embodiment 118

The method of any one of embodiments 91-117, wherein the quantifying the sample level of biomarker is performed with a sensitivity of at least about 80%.

### Embodiment 119

The method of any one of embodiments 91-118, wherein the quantifying the sample level of biomarker is performed with a sensitivity of at least: 80%, 85%, 90%, 91%, 92%, 93%, 94%, 95%, 95%, 97%, 98%, or 99%.

### Embodiment 120

The method of any one of embodiments 91-119, wherein the quantifying the sample level of biomarker is performed with a specificity of at least 90%.

### Embodiment 121

The method of any one of embodiments 91-120, wherein the quantifying the sample level of biomarker is performed with a specificity of at least: 90%, 91%, 92%, 93%, 94%, 95%, 95%, 97%, 98%, or 99%.

### Embodiment 122

The method of any one of embodiments 91-121, further comprising a genetic test.

### Embodiment 123

The method of any one of embodiments 91-122, wherein the genetic test comprises testing for a mutation in a breast cancer susceptibility gene.

### Embodiment 124

The method of any one of embodiments 91-123, wherein the genetic test comprises testing for a mutation in a gene selected from the group consisting of: ATM, BARD1, BRCA1, BRCA2, BRIP1, CASP8, CDH1, CHEK2, CTLA4, CYP19A1, FGFR2, H19, LSP1, MAP3K1, MRE11, NBN, PALB2, PTEN, RAD51, RAD51C, STK11, TERT, TOX3, TP53, XRCC2, XRCC3, and any combination thereof.

### Embodiment 125

The method of any one of embodiments 91-125, wherein the subject has dense breast tissue.

### Embodiment 126

A method for reducing the number of false-positive or false-negative results for a health condition, the method comprising:
a) performing a screening test on a subject, wherein the screening test comprises evaluating the subject for a risk of developing a health condition;
b) obtaining a biological sample of the subject, wherein the subject is from a population of subjects having a positive, negative, or ambiguous result from the screening test;
c) quantifying a sample level of a biomarker in the biological sample of the subject, wherein the biomarker is associated with the health condition;
d) comparing the sample level of the biomarker to a reference level of the biomarker for the health condition; and
e) identifying the result of the screening test as a false-positive or a false-negative for the health condition based on the results of the comparing.

### Embodiment 127

The method of embodiment 126, wherein the health condition is cancer.

### Embodiment 128

The method of any one of embodiments 126-127, wherein the cancer is breast cancer.

### Embodiment 129

The method of any one of embodiments 126-128, wherein the screening test comprises imaging a breast tissue of the subject.

### Embodiment 130

The method of any one of embodiments 126-129, wherein the imaging is performed using a mammogram.

### Embodiment 131

The method of any one of embodiments 126-130, wherein the screening test comprises quantifying a sample level of a cell-free nucleic acid in the subject.

### Embodiment 132

The method of any one of embodiments 126-131, wherein the cell-free nucleic acid is cell-free RNA or cell free DNA.

### Embodiment 133

The method of any one of embodiments 126-132, wherein the cell-free nucleic acid is obtained from a biofluid.

### Embodiment 134

The method of any one of embodiments 121-133, wherein the biofluid is selected from the group consisting of: blood, a blood fraction, serum, plasma, saliva, sputum, urine, semen, a transvaginal fluid, a cerebrospinal fluid, sweat, bile, cyst fluid, tear, breast aspirate, and breast fluid.

### Embodiment 135

The method of any one of embodiments 126-134, wherein the biological sample is a biofluid.

### Embodiment 136

The method of any one of embodiments 126-135, wherein the biofluid is saliva.

### Embodiment 137

The method of any one of embodiments 126-135, wherein the biofluid is blood.

### Embodiment 138

The method of any one of embodiments 126-137, wherein the biofluid is selected from the group consisting of: blood, a blood fraction, serum, plasma, saliva, sputum, urine, semen, a transvaginal fluid, a cerebrospinal fluid, sweat, bile, cyst fluid, tear, breast aspirate, and breast fluid.

### Embodiment 139

The method of any one of embodiments 126-138, wherein the biomarker is selected from the group consisting of: a nucleic acid, peptide, protein, lipid, antigen, carbohydrate and proteoglycan.

### Embodiment 140

The method of any one of embodiments 126-139, wherein the biomarker is a nucleic acid, wherein the nucleic acid is DNA or RNA.

### Embodiment 141

The method of any one of embodiments 126-140, wherein the nucleic acid is RNA, wherein the RNA is selected from the group consisting of: mRNA, miRNA, snoRNA, snRNA, rRNAs, tRNAs, siRNA, hnRNA, and shRNA

### Embodiment 142

The method of any one of embodiments 126-141, wherein the RNA is mRNA.

### Embodiment 143

The method of any one of embodiments 126-141, wherein the RNA is miRNA.

### Embodiment 144

The method of any one of embodiments 126-140, wherein the biomarker is a nucleic acid, wherein the nucleic acid is DNA, wherein the DNA is selected from the group consisting of: double-stranded DNA, single-stranded DNA, complementary DNA, and noncoding DNA.

### Embodiment 145

The method of any one of embodiments 126-144, wherein the biomarker is a cell-free nucleic acid.

### Embodiment 146

The method of any one of embodiments 126-143, wherein the cell-free nucleic acid is a cell free RNA.

### Embodiment 147

The method of any one of embodiments 126-143 and 145-146, wherein the cell-free RNA is cell free mRNA or cell free miRNA.

### Embodiment 148

The method of any one of embodiments 126-147, wherein the biomarker is of exosomal origin.

### Embodiment 149

The method of any one of embodiments 126-148, wherein the biomarker is a gene in a breast cancer pathway.

### Embodiment 150

The method of any one of embodiments 126-149, wherein the biomarker is selected from the group consisting of: LCE2B, HIST1H4K, ABCA2, TNFRSF10A, AK092120, DTYMK, Hs.161434, ALKBH1, MCART1, and any combination thereof.

### Embodiment 151

The method of any one of embodiments 126-150, wherein the biomarker is HIST1H4K.

### Embodiment 152

The method of any one of embodiments 126-150, wherein the biomarker is TNFRSF10A.

### Embodiment 153

The method of any one of embodiments 126-152, wherein quantifying the sample level of the biomarker comprises quantifying at least two biomarkers, wherein the at least two biomarkers are selected from the group consisting of LCE2B, HIST1H4K, ABCA2, TNFRSF10A, AK092120, DTYMK, ALKBH1, MCART1, and Hs.161434.

### Embodiment 154

The method of any one of embodiments 126-153, wherein quantifying the sample level of the biomarker comprises quantifying two biomarkers, wherein the two biomarkers are HIST1H4K and TNFRSF10A.

### Embodiment 155

The method of any one of embodiments 126-154, wherein the subject has dense breast tissue.

### Embodiment 156

The method of any one of embodiments 126-155, further comprising experimentally lysing an exosomal fraction of the biological sample to release the biomarker from the exosomal fraction.

### Embodiment 157

The method of any one of embodiments 126-156, wherein the quantifying further comprises experimentally reverse transcribing the RNA.

### Embodiment 158

The method of any one of embodiments 126-157, wherein the quantifying further comprises performing a polymerase chain reaction.

### Embodiment 159

The method of any one of embodiments 126-158, wherein the wherein the PCR is quantitative PCR.

### Embodiment 160

The method of any one of embodiments 126-159, wherein the quantifying further comprises performing sequencing, wherein the sequencing comprises massively parallel sequencing.

### Embodiment 161

The method of any one of embodiments 126-160, wherein the quantifying the sample level of biomarker is performed with an accuracy of at least 90%.

### Embodiment 162

The method of any one of embodiments 126-161, wherein the quantifying the sample level of biomarker is performed with an accuracy of at least about: 90%, 91%, 92%, 93%, 94%, 95%, 95%, 97%, 98%, 99%, or 99.5%.

### Embodiment 163

The method of any one of embodiments 126-162, wherein the quantifying the sample level of biomarker is performed with a sensitivity of at least about 80%.

### Embodiment 164

The method of any one of embodiments 163, wherein the quantifying the sample level of biomarker is performed with a sensitivity of at least about: 80%, 85%, 90%, 91%, 92%, 93%, 94%, 95%, 95%, 97%, 98%, 99% or 99.5%.

### Embodiment 165

The method of any one of embodiments 126-164, wherein the quantifying the sample level of biomarker is performed with a specificity of at least 90%.

### Embodiment 166

The method of any one of embodiments 126-165, wherein the quantifying the sample level of biomarker is performed with a specificity of at least about: 90%, 91%, 92%, 93%, 94%, 95%, 95%, 97%, 98%, 99%, or 99.5%.

### Embodiment 167

The method of any one of embodiments 126-166, wherein the quantifying the sample level of the biomarker comprises quantifying at least: 2, 3, 4, 5, 6, 7, 8, 9, or 10 biomarkers.

What is claimed is:

1. A method for determining a health state of a subject, the method comprising:
a) providing a saliva sample from a subject;
b) quantifying a sample level of a biomarker from the saliva sample, wherein the biomarker is from an exosome in the saliva sample;
c) comparing the sample level of the biomarker to a reference level of the biomarker, wherein the reference level is obtained from a subject having breast cancer; and
d) determining a risk score of the subject for breast cancer based on the comparing.

2. The method of claim 1, further comprising imaging a breast tissue of the subject.

3. The method of claim 2, wherein the imaging is performed using a mammogram.

4. The method of claim 1, further comprising adjusting the risk score of the subject based on the results from the mammogram.

5. The method of claim 1, further comprising lysing the exosome to release the biomarker prior to step b).

6. The method of claim 4, further comprising enriching an exosome fraction of the saliva sample prior to the lysing.

7. The method of claim 1, further comprising stabilizing the exosome fraction following the enriching.

8. The method of claim 1, wherein the biomarker is a cell-free nucleic acid.

9. The method of claim 8, wherein the cell-free nucleic acid is RNA.

10. The method of claim 9, wherein the RNA is mRNA or miRNA.

11. The method of claim 10, wherein the mRNA is a transcript of a gene selected from the group consisting of LCE2B, HIST1H4K, ABCA1, ABCA2, TNFRSF10A, AK092120, DTYMK, ALKBH1, MCART1, Hs.161434, and any combination thereof.

12. The method of claim 9, wherein the quantifying further comprises reverse transcribing the RNA.

13. The method of claim 1, wherein the quantifying comprises performing a polymerase chain reaction (PCR).

14. The method of claim 13, wherein the PCR comprises qPCR.

15. The method of claim 1, wherein the quantifying further comprises performing sequencing.

16. The method of claim 15, wherein the sequencing comprises massively parallel sequencing.

17. The method of claim 1, wherein the determining the risk score of the subject for breast cancer is performed with an accuracy of at least 90%.

18. The method of claim 1, wherein the determining the risk score of the subject for breast cancer is performed with a specificity of at least 90%.

19. The method of claim 1, wherein the determining the risk score of the subject for breast cancer is performed with a sensitivity of at least 80%.

20. The method of claim 1, wherein the cell-of-origin of the exosome is a breast cell.

21. The method of claim 1, wherein the subject has dense breast tissue.

22. The method of claim 1, wherein the subject has an ambiguous result from a screening mammogram.

23. The method of claim 1, wherein the subject is less than 50 years of age.

24. The method of claim 1, wherein the biomarker is a transcript of a gene associated with a hallmark of cancer.

25. The method of claim 24, wherein the hallmark of cancer is selected from the group consisting of: evading growth suppressor, avoiding immune destruction, promoting replicative immortality, tumor-promoting inflammation, activating invasion and metastasis, inducing angiogenesis, genome instability and mutation, resisting cell death, deregulating cellular energetics, sustaining proliferative signaling, and any combination thereof.

26. The method of claim 24, wherein the gene associated with the hallmark of cancer is selected from the group consisting of: LCE2B, HIST1H4K, ABCA2, TNFRSF10A, AK092120, DTYMK, ALKBH1, MCART1, Hs.161434, and any combination thereof.

27. The method of claim 24, wherein the gene associated with the hallmark of cancer is selected from the group consisting of: ABCA1, ABCA2, TNFRSF10A, DTYMK, ALKBH1, and any combination thereof.

28. The method of claim 1, wherein the biomarker is a transcript of a gene with an expression profile similar to a gene associated with a hallmark of cancer.

29. A method for reducing a number of false-positive or false-negative results for breast cancer, the method comprising:
a) providing a biological sample of a subject, wherein the subject is from a population of subjects having a positive, negative, or ambiguous result from a screening mammogram;
b) quantifying a sample level of a biomarker in the biological sample of the subject;
c) comparing the sample level of the biomarker to a reference level of the biomarker; and
d) identifying the result of the screening mammogram as a false-positive or a false-negative for breast cancer based on the results of the comparing.

30. A method for determining a health state of a subject, the method comprising:
a) providing a biological sample of a subject;
b) quantifying a sample level of at least two biomarkers in the biological sample of the subject, wherein the at least two biomarkers are selected from the group consisting of LCE2B, HIST1H4K, ABCA2, TNFRSF10A, AK092120, DTYMK, ALKBH1, MCART1, Hs.161434, and any combination thereof;
c) comparing the sample level of the at least two biomarkers to a reference level of the two biomarkers; and
d) determining a health state of the subject based on the comparing.

31. A method for determining a health state of a subject, the method comprising:
a) performing a mammogram on a subject;
b) obtaining a saliva sample of the subject;
c) quantifying a sample level of a biomarker from the saliva sample, wherein the biomarker is of exosomal origin, wherein the biomarker is a transcript of a gene selected from the group consisting of: LCE2B, HIST1H4K, ABCA2, TNFRSF10A, AK092120, DTYMK, ALKBH1, MCART1, Hs.161434, and any combination thereof;
d) comparing the sample level of the biomarker to a reference level of the biomarker, wherein the reference level is obtained from a subject having breast cancer; and
e) combining the result of the mammogram and the comparing to determine a health state of the subject associated with breast cancer.

32. A method comprising:
a) providing a saliva sample from a subject;
b) quantifying a sample level of a biomarker from the saliva sample, wherein the biomarker is a transcript of a gene associated with a hallmark of cancer;
c) comparing the sample level of the biomarker to a reference level of the biomarker, wherein the reference level is obtained from a subject having cancer; and
d) determining a risk score of the subject for cancer based on the comparing.

33. The method of claim 32, wherein the hallmark of cancer is selected from the group consisting of: evading growth suppressor, avoiding immune destruction, promoting replicative immortality, tumor-promoting inflammation, activating invasion and metastasis, inducing angiogenesis, genome instability and mutation, resisting cell death, deregulating cellular energetics, sustaining proliferative signaling, and any combination thereof.
